SEQUENCE LISTING (3 sequences)

SEQ ID NO: 1 - DNA, 2118 bp, synthetic construct
  misc_feature 1..2118: note = Description of Artificial Sequence: Synthetic polynucleotide
  source 1..2118: mol_type = other DNA
atggctactcaagatattgacgggcagtatgcaatgactagggctcagagggtccgagctgccatgttccctgagacgctagatgagggcatgcagatcccatccacgcagtttgacgctgctcatcccactaatgtccagcgcttggctgaaccatcacagatgttgaaacatgcagttgtcaatttgattaactatcaggatgacgcggaacttgccacacgtgcaattcctgagctgacaaaactgctaaacgatgaggaccaggtggtagttaataaagctgctgttatggtccatcagctttccaaaaaggaagcttccagacatgccatcatgcgctcccctcagatggtgtctgccattgtacgcaccatgcagaatacaaatgatgtagagacagctcgttgtactgctgggactctgcacaacctttctcaccaccgcgagggcttgctggccatctttaagtctggtggcatcccagcgctggtgaaaatgcttgggtcaccagtggattctgtactgttctacgccatcacgacactgcataatctcctgctccatcaggaaggagctaaaatggcagtgcgcctagctggtggactgcagaaaatggttgctttgctcaacaaaacaaacgtgaaattcttggctattacaacagactgccttcagatcttagcttatggcaatcaagagagcaagctcatcattctggccagtggtggaccccaagccttagtaaacataatgaggacctacacttatgagaagcttctgtggaccacaagcagagtgctgaaggtgctgtctgtctgctctagcaacaagccggccattgtagaagctggtgggatgcaggcactggggcttcatctgacagacccaagtcagcgacttgttcaaaactgtctttggactctcagaaacctttcagatgcagcgactaagcaggaagggatggaaggcctccttgggactctagtgcagcttctgggttccgatgatataaatgtggtcacctgtgcagctggaattctctctaacctcacttgcaataattacaaaaacaagatgatggtgtgccaagtgggtggcatagaggctcttgtacgcaccgtccttcgtgctggtgacagggaagacatcactgagcctgccatctgtgctcttcgtcatctgaccagccggcatcaggaagccgagatggcccagaatgccgttcgccttcattatggactgcctgttgtggttaaactcctgcacccaccatcccactggcctctgataaaggcaactgttggattgattcgaaaccttgccctttgcccagcaaatcatgcgcctttgcgggaacagggtgctattccacgactagttcagctgcttgtacgagcacatcaggacacccaacggcgcacctccatgggtggaacgcagcagcagtttgtggagggcgtgcgcatggaggagatagtagaagggtgtactggagctctccacatccttgctcgggacgttcacaaccggattgtaatccgaggactcaataccattccattgtttgtgcagttgctttattctcccattgaaaatatccaaagagtagctgcaggggtcctctgtgaacttgctcaggacaaggaggctgcagaggccattgaagctgagggagccacagctcccctgacagagttactccactccaggaatgaaggcgtggcaacatacgcagctgctgtcctattccgaatgtctgaggacaagccacaggattacaagaagcggctttcagtcgagctgaccagttccctcttcaggacagagccaatggcttggaatgagactgcagatcttggactggacattggtgcccagggagaagcccttggatatcgccaggatgatcccagctaccgttcttttcactctggtggatacggccaggatgccttggggatggaccctatgatggagcatgagatgggtggccaccaccctggtgctgactatccagttgatgggctgcctgatctgggacacgcccaggacctcatggatgggctgcccccaggtgatagcaatcagctggcctggtttgatactgacctgtaa

SEQ ID NO: 2 - DNA, 2118 bp, synthetic construct
  misc_feature 1..2118: note = Description of Artificial Sequence: Synthetic polynucleotide
  source 1..2118: mol_type = other DNA
atggctactcaagatatcgacgggcagtatgcaatgaccagggctcagcgagtccgagctgccatgttcccagagaccctagatgagggaatgcagatcccatccacgcagtttgacgctgctcatcccactaatgtccagcgtcttgctgaaccatcccagatgttgaaacatgctgttgtcaatcttattaactatcaggatgacgcagaacttgccacaagagcaattcctgaactgacaaaactgctaaacgatgaggaccaggtggtcgttaataaggctgctgttatggtccatcagctttccaaaaaggaagcttccagacatgctatcatgagatcccctcagatggtgtctgccattgtacgtaccatgcagaatacaaatgatgttgagaccgctcgttgtactgctggtactctgcacaacctttctcaccaccgcgaaggactgctggccatctttaaatctggtggaatccctgctctggtcaaaatgctgggatcacctgtcgattctgtactgttctacgccattacaacactgcataatctcctgctccaccaggaaggagcaaagatggctgtgcgcctagctggtggactgcagaaaatggtggctttgctcaacaaaacaaatgtgaagtttttggctattacgacggactgcctgcagatcttagcttacggaaatcaagagtctaagctcatcattctggcctctggaggaccccaagccttagtcaacatcatgaggacctacacttatgagaagcttctgtggacaaccagcagagtgctgaaggtgctgtctgtctgctctagcaataagccggccattgtagaagctggagggatgcaggcactgggactgcatctgacagatccatcccagcgacttgtgcagaactgtctttggactctcagaaatctttccgatgcagcgactaagcaagaagggatggaaggcctcctggggactctggttcagcttctgggttccgatgatataaatgtggtcacctgcgcagctggaattctctctaacctcacctgcaataattacaaaaataagatgatggtgtgccaagtgggtggcatagaggctctggtacgcaccgtccttcgtgctggtgatcgtgaagacatcactgagcctgcgatctgtgctctgcgtcacctgaccagccggcatcaggaagcagagatggcccagaatgctgttcgccttcattacggtctgcctgttgtggttaaacttctgcacccaccttctcactggcccctgataaaggcaactgttggattgattcgaaaccttgctctttgcccagctaatcatgctcctctgcgagaacagggagctatcccacgactggttcagcttcttgtccgtgctcatcaggacacccaacgtcgcacctccatgggtggaacgcagcagcaatttgttgagggcgtgagaatggaggagatcgtggaagggtgtaccggagctctccacatcctcgctcgagacgtccacaaccggatcgtaatcagaggacttaataccattccattgtttgtgcaactgctttactcgcctattgaaaacatccaaagagtcgctgctggggtcctctgtgaacttgctcaggataaggaggctgccgaggccattgaagctgagggagctaccgctcctctgacagagttactgcactcccgtaatgagggagtcgcaacctacgcagctgcagtcctgttccgaatgtctgaggacaagccacaggattacaagaagcggctgtccgttgagctcaccagttccctcttccgtaccgagccaatggcttggaacgagactgcagatcttggactggatattggtgcccagggagaagcccttggatatcgtcaggacgaccccagctaccgttcttttcattctggtggatacggtcaggatgccttggggatggaccctatgatggagcatgaaatgggtggccatcaccctggagctgactatccagttgatggactgcctgatctgggacatgcccaggacctcatggatgggctgcccccaggtgattcaaatcagctcgcctggttcgatactgacctgtaa

SEQ ID NO: 3 - protein, 705 aa, synthetic construct
  REGION 1..705: note = Description of Artificial Sequence: Synthetic polypeptide
MATQDIDGQYAMTRAQRVRAAMFPETLDEGMQIPSTQFDAAHPTNVQRLAEPSQMLKHAVVNLINYQDDAELATRAIPELTKLLNDEDQVVVNKAAVMVHQLSKKEASRHAIMRSPQMVSAIVRTMQNTNDVETARCTAGTLHNLSHHREGLLAIFKSGGIPALVKMLGSPVDSVLFYAITTLHNLLLHQEGAKMAVRLAGGLQKMVALLNKTNVKFLAITTDCLQILAYGNQESKLIILASGGPQALVNIMRTYTYEKLLWTTSRVLKVLSVCSSNKPAIVEAGGMQALGLHLTDPSQRLVQNCLWTLRNLSDAATKQEGMEGLLGTLVQLLGSDDINVVTCAAGILSNLTCNNYKNKMMVCQVGGIEALVRTVLRAGDREDITEPAICALRHLTSRHQEAEMAQNAVRLHYGLPVVVKLLHPPSHWPLIKATVGLIRNLALCPANHAPLREQGAIPRLVQLLVRAHQDTQRRTSMGGTQQQFVEGVRMEEIVEGCTGALHILARDVHNRIVIRGLNTIPLFVQLLYSPIENIQRVAAGVLCELAQDKEAAEAIEAEGATAPLTELLHSRNEGVATYAAAVLFRMSEDKPQDYKKRLSVELTSSLFRTEPMAWNETADLGLDIGAQGEALGYRQDDPSYRSFHSGGYGQDALGMDPMMEHEMGGHHPGADYPVDGLPDLGHAQDLMDGLPPGDSNQLAWFDTDL

(19) United States
(12) Patent Application Publication
(10) Pub. No.: US 2024/0316091 A1
(43) Pub. Date: Sep. 26, 2024

(54) MCM FOR GENE THERAPY TO ACTIVATE WNT PATHWAY

(71) Applicant: STEADMAN PHILIPPON RESEARCH INSTITUTE, Vail, CO (US)

(72) Inventors: Chelsea BAHNEY, Vail, CO (US); Ralph MARCUCIO, San Francisco, CA (US); Gianluca FONTANA, Madison, WI (US); William L. MURPHY, Madison, WI (US); John P. COOKE, Houston, TX (US); Daniel L. KISS, Houston, TX (US); Francesca TARABALLI, Houston, TX (US); Nicole EHRHART, Fort Collins, CO (US); Anna-Laura NELSON, Leadville, CO (US)

(21) Appl. No.: 18/680,866

(22) Filed: May 31, 2024

Related U.S. Application Data
(63) Continuation of application No. 17/683,699, filed on Mar. 1, 2022.
(60) Provisional application No. 63/155,263, filed on Mar. 1, 2021.

(51) Int. Cl. A61K 31/7105; A61P 19/00; C07H 21/02

(52) U.S. Cl. CPC A61K 31/7105; A61P 19/00; C07H 21/02

(57) ABSTRACT

The present disclosure is related to methods of stimulating bone formation for the purpose of improving bone repair, accelerating bone healing, and/or generating now bone in a local region with absent or diminished bone due to injury, disease, or defect, comprising administering a composition comprising β-catenin mRNA complex to the subject.

### CROSS-REFERENCE TO RELATED APPLICATIONS

This application is a continuation of U.S. patent application Ser. No. 17/683,699, filed on Mar. 1, 2022, which claims priority to U.S. Provisional Patent Application No. 63/155,263, filed Mar. 1, 2021, the entire content of each of which is incorporated by reference herein.

### GOVERNMENT LICENSE RIGHTS

This invention was made with government support under TR002383 awarded by the National Institutes of Health. The government has certain rights in the invention.

### FIELD OF THE DISCLOSURE

The present disclosure is related to methods of accelerating fracture repair in a subject, comprising administering a composition comprising β-catenin mRNA complex bound to mineral coated microparticles (MCM) to the subject.

### SEQUENCE LISTING

The instant application contains a Sequence Listing, which has been submitted electronically in XML format and is hereby incorporated by reference in its entirety. Said XML copy, created on May 29, 2024, is named 18472-0015USC1_SeqListing.xml and is 10,240 bytes in size.

### BACKGROUND OF THE DISCLOSURE

Bone fractures are one of the most common injuries worldwide. Complication in fracture healing, such as delayed or non-union, are estimated to occur in approximately 10-15% of healthy individuals (Giannoudis, et al. 2005 Injury 36 S3:S20-27). However, impaired healing rates approach 50% following high-velocity injuries or in individuals with high co-morbidities, including, diabetes, obesity, aging, estrogen deficiency, malnutrition, and smoking (Hellwinkel and Miclau 2020 JBJS Rev 8:e1900221). The Lancet Commission named the treatment of open fractures as one of the three highest value surgical procedures to improve global health, based on their propensity to drive problematic healing and the huge impact this creates on patient quality of life and healthcare cost burden (Meara, et al. 2015 Lancet 386:569-624; Bagguley, et al. 2019 BMJ Open 9:e029812; O'Neill, et al. 2011 Spine J 11:641-646). There thus remains an unmet clinical need for approaches to augment fracture repair.

The current standard of care to treat poorly healing fractures is surgical intervention to increase biomechanical stability or promote healing through application of bone grafts. Bone autograft remains the gold standard clinical technique for augmenting bone healing in these cases. While autograft is associated with good healing outcomes, bone harvest increases surgical time and risk of complications by ˜60%, is associated with a high incidence of donor site morbidity, and there is insufficient bone available to fill large defects. Bone allograft is readily available in a number of form factors, but product failure rates are reported between 20-40% (Enneking and Campanacci 2001 J Bone Joint Surg, Amer Vol 83-A:971-986; Wheeler and Enneking 2005 Clin orthopaed related res 36-42). To avoid the risks of surgery in elderly patients, they are often monitored for up to 1 year prior to surgical intervention. Consequently, elderly fracture patients often suffer long recovery times, leading to increased frailty, depression, and loss of independence with progressive complications increasing morbidity. Developing and validating non-surgical therapeutics to stimulate bone regeneration could improve clinical options and outcomes in fracture healing. However, there are no FDA-approved pharmacologics to accelerate fracture repair or treat non-union (Kostenuik and Mirza 2017 J Orthopaed res:offic pub Orthopaed Res Soc 35:213-223). As such, there is also an unmet clinical need for therapeutics that stimulate fracture healing through a non-surgical delivery platform.

Bones are one of the few organs with true regenerative potential. The healing process replicates embryonic development programs to form bone indirectly from a cartilage template through the process of endochondral ossification (Bahney, et al. 2019 J orthopaed res:office pub Orthopaed Res Soc 37:35-50). Significant progress has been made in recent years to advance the understanding of the cellular and molecular mechanisms of endochondral ossification. Recent work has demonstrated that chondrocytes become the osteoblasts that give rise to the new bone (Bahney, et al. 2014 J Bone Miner Res 29:1269-1282). However, most therapeutics under investigation for fracture healing aim to promote direct, or intramembranous, bone repair (Almubarak, et al. 2015 Bone 83:197-209). This disconnect between current therapies and the endogenous mechanism of fracture repair represents a potential explanation for poor or inconsistent outcomes with existing osteoinductive therapeutics.

Bone morphogenetic proteins (BMPs) are the most widely recognized osteoinductive protein with a clinical product, INFUSER, that combines BMP2 onto a surgically implanted collagen sponge. INFUSER has FDA approval within a narrow indication of tibial fractures, but widespread off label use was once reported. Clinical use of BMP has fallen out of favor due to the high cost, limited evidence of clinical efficacy, and severe off-target effects (Benglis, et al. 2008 Neurosurg 62:ONS423-431; Carragee, et al. 2011 The Spine J:office J N Am Spine Soc 11:471-491; Tannoury and An 2014 The Spine J:office J N Am Spine Soc 14:552-559). Recently, a number of systemic osteoanabolic drugs designed to prevent osteoporotic fractures have also come onto the market by acting on the parathyroid (FORTEO®, TYMLOS®) or Wnt (EVENITY®, PROTELOS®) pathways. While each has some preclinical evidence for enhanced fracture healing in rodent models, to-date, there is no evidence of a clinical benefit (Schemitsch, et al. 2020 J Bone Joint Surg, Amer vol 102:693-702; Bhandari, et al. 2020 J Bone Joint Surg, Amer vol 102:1416-1426).

Another osteoinductive program, Wnt signaling is categorized according to the β-catenin-dependent canonical pathway and the β-catenin-independent non-canonical pathways. While some evidence suggests that the non-canonical pathways may play a role in regulating osteogenesis, the canonical Wnt/β-catenin pathway is well established for its role promoting bone formation and intramembranous bone repair (Monroe, et al. 2012 Gene 492:1-18; Schupbach, et al. 2020 Bone 138:115491; Wong, et al. 2018 Front Bioeng Biotechnol 6:58; Grigoryan, et al. 2008 Genes & Dev 22:2308-2341). Limited research has been done to determine the role of canonical Wnt signaling during endochondral bone formation and repair. In recent years, there has been a rapid expansion and validation of research demonstrating that chondrocytes become osteoblasts during endochondral bone development (Yang, et al. 2014 PNAS USA 1302703111) and repair (Wong, et al. 2020 J orthopaed res:office pub Orthopaed Res Soc 24904). Current data suggests that the canonical Wnt pathway acts as a key “molecular switch” required for chondrocyte to osteoblast transformation (Wong, et al. 2020 bioRxiv 2020.2003.2011.986141).

Several modulators of the canonical Wnt pathway have been tested in preclinical and clinical models. Lipid modification of Wnt ligands is required to enable intracellular trafficking and pathway activation. As such, simple manufacturing and delivery of a recombinant Wnt ligands is not economical (Takada, et al. 2006 Dev Cell 11:791-801). The majority of commercial strategies utilize neutralizing antibodies to pathway inhibitors to indirectly activate Wnt signaling. Alternatively, the natural elements fluoride and strontium have been shown to activate Wnt signaling. Fluoride works by blocking the activity of the destruction complex and decreasing the secretion of Wnt inhibitors. Strontium has been shown to simultaneously increase bone formation and decrease bone resorption, acting on the Wnt pathway by decreasing the expression of sclerostin and increasing the expression of Wnt3a and Wnt11. However, to date, these Wnt activating approaches have either not been effective (Schemitsch, et al. 2020 J Bone Joint Surg, Amer vol 102:693-702; Bhandari, et al. 2020 J Bone Joint Surg, Amer vol 102:1416-1426) or not tested for their ability to accelerate fracture repair, and alternative approaches are needed to create highly bioactive and localized Wnt-activating therapies.

### BRIEF SUMMARY OF THE DISCLOSURE

In one aspect, the disclosure provides a method of stimulating bone formation for the purpose of improving bone repair, accelerating bone healing, and/or generating new bone in a local region with absent or diminished bone due to injury, disease, or defect, comprising administering a composition comprising β-catenin mRNA complex to the subject. In one aspect, the disclosure provides a method of stimulating bone healing, accelerating bone healing, and/or improving bone healing in a subject, comprising administering a composition comprising β-catenin mRNA complex to the subject. By β-catenin mRNA “complex,” it is meant that the mRNA is complexed with a stabilizing/delivery agent. In one embodiment, the bone healing is bone fracture healing. In one embodiment of a method according to the disclosure, the subject has normal bone healing. In another embodiment, the subject has delayed or non-union bone healing.

In another aspect, the disclosure provides a method for accelerating fracture repair in a subject, comprising administering a composition comprising β-catenin mRNA complex to the subject.

In another aspect, the disclosure provides a method of treating malunion, delayed union, or non-union in a subject, comprising administering a composition comprising β-catenin mRNA complex to the subject.

In one embodiment of a method according to the disclosure, bone regeneration is stimulated in the subject. “Stimulated,” as used herein, means promoted or enhanced. In another embodiment, the bone regeneration is within a bone fracture site in the subject.

In another aspect, the disclosure provides a method for stimulating bone regeneration in a subject, comprising administering a composition comprising β-catenin mRNA complex to the subject.

In one embodiment of a method according to the disclosure, the Wnt signaling pathway is activated in the subject. The term “activated,” as used in the instant context, means turned on.

In one embodiment of a method according to the disclosure, the β-catenin mRNA (of the complex) is a non-destructible β-catenin mRNA. “Non-destructible,” as used herein, refers to a mRNA sequence that will produce a modified β-catenin protein that cannot be phosphorylated and/or ubiquitinated and targeted for subsequent proteasomal degradation. Similarly, this modification can be referred to as a β-catenin mRNA with a gain-of-function mutation. The “non-destructible” or “Gain-of-function” (“GOF”)-catenin protein results in the downstream activation of the canonical Wnt signaling pathway.

In one embodiment of a method according to the disclosure, one or more codons of the β-catenin^{GOF} mRNA are modified to: i) optimize stability and/or translatability of the mRNA; and/or ii) reduce immunogenicity of the mRNA.

In one embodiment of a method according to the disclosure, the β-catenin mRNA (of the complex) is circular. In another embodiment, the β-catenin mRNA is linear.

In one embodiment of a method according to the disclosure, the β-catenin mRNA complex is encapsulated in a lipidic transfecting agent. In another embodiment, the lipidic transfecting agent is a lipid nanoparticle. In still another embodiment, the lipid nanoparticle comprises a combination of an organic phase and an aqueous phase, wherein the organic phase comprises lipids in ethanol. In a further embodiment, the lipids are DLin-MC3, DSPC, Cholesterol, and DMG-PEG. In still a further embodiment, the lipids DLin-MC3, DSPC, Cholesterol, and DMG-PEG are at a ratio of about 50:about 10.5:about 38:about 1.5.

In one embodiment of a method according to the disclosure, the β-catenin mRNA complex is bound to mineral coated microparticles (MCM). In another embodiment, the mRNA is encapsulated in a lipidic transfecting agent, and the resulting complex is bound to MCM. In still another embodiment, the mRNA itself is bound to MCM.

In one embodiment of a method according to the disclosure, the MCM are spherical or rod-shaped. In another embodiment, the MCM are biocompatible. In still another embodiment, the MCM are biodegradable.

In one embodiment of a method according to the disclosure, the MCM comprise a mineral coating comprising Ca²⁺ and/or PO₄³⁻. In another embodiment, the MCM comprise a mineral coating comprising at least one chemical dopant. In still another embodiment, the at least one chemical dopant is fluoride or strontium. The chemical doping of the MCM may improve transfection of the β-catenin mRNA.

In one embodiment of a method according to the disclosure, the MCM are entrapped on a biodegradable scaffold. In another embodiment of a method according to the disclosure, the MCM are entrapped on hydrogel. In another embodiment, the hydrogel is alginate.

In one embodiment of a method according to the disclosure, the composition further comprises an osteoconductive graft. In another embodiment, the osteoconductive graft is selected from the group consisting of an autograft, an allograft, demineralized bone matrix, and a collagen scaffold.

In one embodiment of a method according to the disclosure, the composition is administered to the subject via injection. In another embodiment, the composition is administered via subcutaneous or percutaneous injection. In still another embodiment, the composition is injected locally into the subject. By “locally” is meant directly to the site in which bone healing and/or bone regeneration is desired. In still another embodiment, the composition is injected into and/or adjacent to a bone defect of the subject. The phrase “bone defect,” as used herein, refers to a bone gap, a segmental bone defect, a bone crack, a fracture callus, a necrotic bone, and/or localized osteopenia.

In one embodiment of a method according to the disclosure, the subject has a bone fracture, and the composition is administered during the intramembranous periostal repair phase or at the end of the endochondral repair phase of fracture healing. In another embodiment, the subject has a bone fracture, and the composition is administered during the intramembranous periostal repair phase and at the end of the endochondral repair phase of fracture healing. In still another embodiment, the subject has a bone fracture, and the composition is administered following acute inflammation to promote the initial periosteal healing response or to the soft callus phase of healing to promote endochondral repair.

In one embodiment of a method according to the disclosure, the β-catenin mRNA complex is gradually released from the MCM upon administration of the composition. In another embodiment of a method according to the disclosure, canonical Wnt signaling is activated upon administration of the composition. In still another embodiment of a method according to the disclosure, administration of the composition results in endochondral conversion of cartilage to bone.

In one aspect, the disclosure provides a composition comprising β-catenin mRNA complex.

In one embodiment of a composition according to the disclosure, the β-catenin mRNA is a non-destructible β-catenin mRNA. In another embodiment of a composition according to the disclosure, the β-catenin mRNA has a gain-of-function mutation. In another embodiment, one or more codons of the β-catenin^{GOF} mRNA are modified to: i) optimize stability and/or translatability of the mRNA; and/or ii) reduce immunogenicity of the mRNA.

In one embodiment of a composition according to the disclosure, the β-catenin mRNA is circular. In another embodiment, the β-catenin mRNA is linear.

In one embodiment of a composition according to the disclosure, the β-catenin mRNA complex is encapsulated in a lipidic transfecting agent. In another embodiment, the lipidic transfecting agent is a lipid nanoparticle. In still another embodiment, the lipid nanoparticle comprises a combination of an organic phase and an aqueous phase, wherein the organic phase comprises lipids in ethanol. In a further embodiment, the lipids are DLin-MC3, DSPC, Cholesterol, and DMG-PEG. In still a further embodiment, the lipids DLin-MC3, DSPC, Cholesterol, and DMG-PEG are at a ratio of about 50:about 10.5:about 38:about 1.5.

In one embodiment of a composition according to the disclosure, the β-catenin mRNA complex is bound to mineral coated microparticles (MCM).

In one embodiment of a composition according to the disclosure, the MCM are spherical or rod-shaped. In another embodiment, the MCM are biocompatible. In still another embodiment, the MCM are biodegradable.

In one embodiment of a composition according to the disclosure, the MCM comprise a mineral coating comprising Ca²⁺ and/or PO₄³⁻. In another embodiment, the MCM comprise a mineral coating comprising at least one chemical dopant. In still another embodiment, the at least one chemical dopant is fluoride or strontium.

In one embodiment of a composition according to the disclosure, the MCM are entrapped on a biodegradable scaffold. In another embodiment of a composition according to the disclosure, the MCM are entrapped on hydrogel. In another embodiment, the hydrogel is alginate.

In one embodiment, a composition according to the disclosure further comprises an osteoconductive graft. In another embodiment, the osteoconductive graft is selected from the group consisting of an autograft, an allograft, demineralized bone matrix, and a collagen scaffold.

In certain embodiments, a pharmaceutical composition according to the disclosure further comprises at least one pharmaceutically acceptable excipient or carrier.

In one embodiment, a composition according to the disclosure is formulated for administration via injection. In another embodiment, the composition is formulated for subcutaneous or percutaneous injection.

In certain embodiments, a composition according to the disclosure is for use in stimulating bone healing, accelerating bone healing, and/or improving bone healing in a subject. In one embodiment, the bone healing is bone fracture healing.

In one embodiment, a composition according to the disclosure is for use in accelerating fracture repair in a subject.

In another embodiment, a composition according to the disclosure is for use in treating malunion in a subject. In still another embodiment, the malunion is delayed union or non-union.

In another embodiment, a composition according to the disclosure is for use in stimulating bone regeneration in a subject. In another embodiment, the bone regeneration is within a bone fracture site in the subject.

In one embodiment, administration of a composition according to the disclosure results in activation of the Wnt signaling pathway in the subject.

In an additional embodiment, a method or composition according to the disclosure is useful in osteoporotic indications. In a further embodiment, the osteoporotic indication is osteoporotic fracture. In a still further embodiment, the osteoporotic fracture is atypical femoral neck fracture.

In an additional embodiment, a method or composition according to the disclosure is useful in craniofacial indications. In a further embodiment, the craniofacial indication is selected from the group consisting of craniostenosis/craniosynostosis, cleft palate, mandibular fracture, cranial bone fracture, and cranial bone defect.

Other embodiments will become apparent from a review of the ensuing detailed description.

### BRIEF DESCRIPTION OF THE FIGURES

FIG. 1 shows a schematic illustration of the phases and timeline for endochondral fracture repair in a murine model of tibia fracture.

FIG. 2 shows a schematic diagram of mineral coated microparticles (MCM) for delivery of protein.

FIGS. 3A-3G show chondrocyte characterization after treatment with MCM and FMCM. Per (FIG. 3A) Presto Blue quantification of chondrocytes treated with 0-250 μg of MCM show no cytotoxic effect. Temporal gene expression of chondrocytes treated with 12.5 μg/well MCM or FMCM shows that (FIG. 3B) MCM stimulates osteocalcin expression from 3-24 hrs, and that FMCM significantly activate downstream Wnt genes (FIG. 3C) axin2 and (FIG. 3D) Cntb1. (n=3-4, *p<0.05, *<0.01, ***<0.001). (FIG. 3E) shows the levels of secreted alkaline phosphatase compared between treatments. (FIG. 3F) shows the qRT-PCR results for osteopontin (Opn) compared between treatments. (FIG. 3G) shows cell viability following MCM and FMCM treatment.

FIGS. 4A-4D show temporal gene expression of (FIG. 4A) firefly luciferase in ATDC5 chondrocytes delivered with lipofectamine alone, MCM, or FMCM. (FIG. 4B) shows firefly RNA expression without the log transformation analysis results shown in FIG. 4A. (FIG. 4C) shows temporal expression of IL1β in ATDC5 chondrocytes delivered with lipofectamine alone, MCM, or FMCM. (FIG. 4D) shows firefly luciferase expression (mRNA expression) at 3 hr, 6 hr, 24 hr, 48 hr, and 72 hr timepoint for non-transfected (NT) chondrocyte cells, as well as chondrocytes transfected with mRNA with lipid nanoparticles (LNP), with mRNA with LNP-MCM, and with LNP-FMCM.

FIGS. 5A-5E show (FIG. 5A) Pin-stabilized tibia fracture, (FIG. 5B) Intra-callus injections, (FIG. 5C) IVIS imaging days 7-13 post fracture (1-7 post injection) of MCM only, mRNA only, or mRNA-MCM; (FIG. 5D) Semi-quantification of IVIS; (FIG. 5E) FFLuc expression in FRX callus.

FIGS. 6A-6Q show that activating canonical Wnt with β-cat^{GOF} significantly increases bone formation and accelerates fracture repair. (FIGS. 6A, 6C, 6E, 6G, 6I, 6K—wild-type, 6B, 6D, 6F, 6H, 6J, 6L—GOF) Hall Brundt's Quadruple stain (HBQ histology) shows increased bone formation (red) and decreased cartilage (blue) in the fracture calli at all times during repair. (FIG. 6M) Axin2 gene expression is upregulated by β-cat^{GOF} d10 post-fracture, fracture callus. (FIG. 6N: total callus, FIG. 6O: % bone, FIG. 6P: % cartilage, FIG. 6Q: % marrow) Histomorphometric quantification confirms increased bone and decreased cartilage composition in fracture callus. N=5/group/time, Scale=1000 μm. (*)=p<0.05. (**)=p<0.01.

FIG. 7 shows a schematic illustration of a circβ-cat^{GOF} mRNA.

FIGS. 8A and 8B show the temporal gene expression of (FIG. 8A) firefly luciferase and (FIG. 8B) IL1β in ATDC5 chondrocytes treated with 25 μg of luciferase mRNA encapsulated in lipofectamine or engineered lipid nanoparticles (LNPs) relative to negative controls. (n=3-4, *p<0.05, **<0.01)

FIGS. 9A-9D show the temporal expression of osteogenic and angiogenic genes following (FIG. 9A (timeline), FIG. 9B (relative expression graph)): intramembranous/early delivery of NGF, or (FIG. 9C (timeline), FIG. 9D (relative expression graph)): late/endochondral delivery of NGF. (n=3-4, *p<0.05, **<0.01)

### DETAILED DESCRIPTION

Before the present methods are described, it is to be understood that this disclosure is not limited to particular methods, and experimental conditions described, as such methods and conditions may vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, preferred methods and materials are now described. All publications mentioned herein are incorporated herein by reference in their entirety.

### Definitions

The term “fracture” or “bone fracture”, as used herein, refers to a partial or complete break in the continuity of a bone. The fracture of the bone may be closed or open (compound). The fracture of the bone may be displaced. Stress fractures, also referred to as hairline fractures, are also bone fractures. Bone fractures may be transverse, spiral, oblique, compression, comminuted, avulsion, impacted, etc. A bone fracture may be diagnosed via X-ray imaging, magnetic resonance imaging (MRI), bone scan, computed tomography scan (CT/CAT scan), or other known methods.

Bone fracture treatment traditionally depends on the location, type, and severity of fracture. Treatment may include repositioning the bone, followed by immobilization via a plaster or fiberglass cast, repositioning the bone, followed by partial immobilization via a functional cast or brace, support/partial immobilization via splint, open reduction with internal fixation, open reduction with external fixation, and other methods known to the clinician.

By the phrase “therapeutically effective amount” is meant an amount that produces the desired effect for which it is administered. In one embodiment, a therapeutically effective amount is an amount that increases the rate and/or amount of bone formation. In certain embodiments, clinical determination that a bone is healing better and/or that more bone has formed is based on one or more of: (1) X-ray, (2) computerized/computed tomography (CT), (3) reduced pain, (4) reduced mobility, and (5) elevated biomarkers, such as, alkaline phosphates, bone-specific alkaline phosphatase, P1NP, CTX, collagen (type) 10. In other embodiments, a non-clinical determination that a bone is healing better and/or that more bone has formed is based on one or more of: activation of Wnt signaling at a gene or protein level, bone healing measured by histology and/or CT (for example, more bone and less cartilage), and biomarkers.

The exact amount will depend on the purpose of the treatment, and will be ascertainable by one skilled in the art using known techniques (see, for example, Lloyd (1999), The Art, Science and Technology of Pharmaceutical Compounding). In certain embodiments, a composition according to the disclosure or for use (for example, in a method) according to the disclosure, comprises a therapeutically effective amount of each of a β-catenin mRNA (for example, β-catenin^{GOF} mRNA, lipidic transfecting agent, and/or mineral coated microparticle.

As used herein, the term “subject” refers to an animal, preferably a mammal, more preferably a human. As such, subjects of the disclosure may include, but are not limited to, humans and other primates, such as chimpanzees and other apes and monkey species; farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys, and other gallinaceous birds, ducks, geese, and the like. In certain embodiments, the subject is a human. The term includes mammalian, including human, subjects having a bone defect or fracture and/or needing bone regeneration.

As used herein, the terms “treat”, “treating”, or “treatment” refer to the healing of a bone fracture in a subject in need thereof. The terms include healing of the actual fracture and may additionally or alternatively include ameliorating a symptom associated with the bone fracture, for example, pain, inflammation, reduced mobility, etc. The terms “treat”, “treating”, or “treatment” also refer to stimulating bone regeneration in a subject in need thereof.

### Fracture Healing

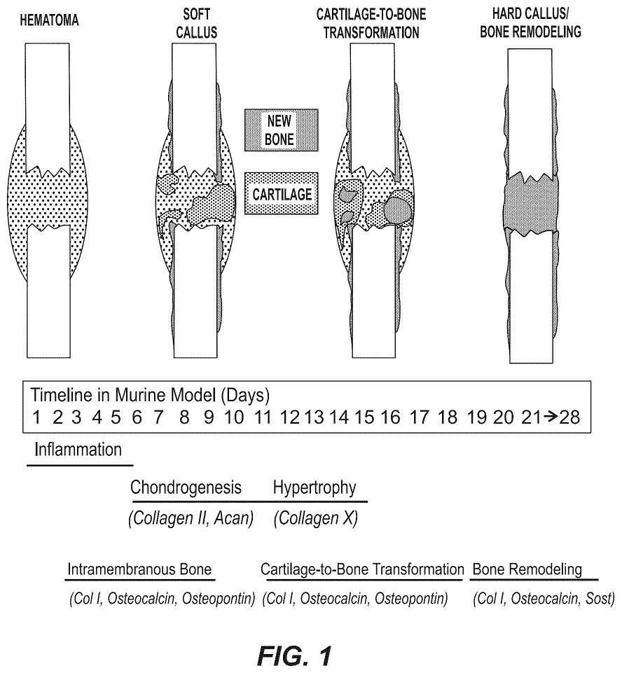

Fracture healing is a dynamic regenerative process that can fully restore the native form and function of an injured bone. The majority of fractures heal indirectly through a cartilage intermediate in a process that draws parallels to endochondral ossification (EO) during long bone formation (FIG. 1). Following a long bone fracture, a hematoma forms to stop the bleeding, contain debris, and trigger a pro-inflammatory response that initiates repair (Kolar, et al., 2010, Tissue Engineering, Part B, Reviews 16:427-434; Xing, et al., 2010, J Orthopaedic Res 28:1000-1006). Periosteal and endosteal progenitor cells undergo osteogenic differentiation to form new bone along the existing bone ends adjacent to the fracture through intramembranous ossification (Colnot, et al., 2009, J Bone Miner Res 24:274-282). In the fracture gap, periosteal progenitor cells differentiate into chondrocytes and generate a provisional cartilaginous matrix that gives rise to bone indirectly by EO (Le, et al., 2001, J Orthopaed Res 19:78-84). The cartilage callus matures to bone through transformation of chondrocytes into osteoblasts (Hu, et al., 2017, Development 144:221-234; Zhou, et al., 2014, PloS genetics 10:e1004820; Yang, et al., 2014, PNAS USA 1302703111). The newly formed trabecular bone then remodels into cortical bone (Drissi, et al., 2016, J Cellular Biochem 117:1753-1756).

Bone fracture healing comprises an inflammatory phase (fracture hematoma formation), a repairing/reparative phase (during which the body develops cartilage and tissue in and around the fracture site, calluses grow and stabilize the fracture, and trabecular bone replaces the tissue callus), and a bone remodeling phase (during which spongy bone is replaced with solid bone). During the inflammation stage of fracture healing/repair, the biological processes hematoma, inflammation, and recruitment of mesenchymal stem cells take place. During the cartilage formation and periostal response stage of fracture healing/repair, the biological processes chondrogenesis and endochondral ossification, cell proliferation in intramembranous ossification, vascular in-growth, and neo-angiogenesis occur. During the cartilage resorption and primary bone formation stage of fracture healing/repair, the biological processes active osteogenesis, bone cell recruitment and woven bone formation, chondrocyte apoptosis and matrix proteolysis, osteoclast recruitment and cartilage resorption, and neo-angiogenesis take place. Finally, during the secondary bone formation and remodeling stage of fracture healing/repair, the biological processes bone remodeling coupled with osteoblast activity and establishment of marrow occur (Al-Aql, et al., 2008, J Dent Res 87(2):107-118).

Thus, in one embodiment of a method or composition according to the disclosure, bone healing comprises the formation of new bone, wherein newly formed bone contains higher trabecular number, connective density, and/or bone mineral density. In another embodiment, bone healing comprises a decrease in cartilage volume in the subject and an increase in bone volume in the subject. In yet another embodiment, the cartilage volume in the subject decreases by at least about 10%, and bone volume in the subject increases by at least about 10% upon administration of the composition. In still another embodiment, cartilage volume in the subject decreases by at least about 25%, and bone volume in the subject increases by at least about 25% upon administration of the composition. In specific embodiments, the % decrease in cartilage volume and/or % increase in bone volume is local (to the treatment).

In certain embodiments, a subject does not experience normal fracture healing. In specific embodiments, such a subject may experience mal-union (bone fracture healing in a deformed, non-anatomical position; can be functionally and/or cosmetically unacceptable), delayed (significantly longer, for example, about twice as long as expected/average fracture healing time), or non-union (failure of the broken bones to unite) fracture healing.

Average fracture healing time may differ depending on the specific bone and/or the level of blood supply in the area of the bone. For example, fractures present in areas of high blood supply, like the spine, the wrist, etc., heal earlier than fractures present in areas of low blood supply, like the scaphoid (wrist bone), the tibia (leg bone), etc. Average fracture healing time may also vary depending on the age of the subject, where the same bone fracture may take twice as long to heal in an elderly person as in a child. The clinician is aware of the general ranges of healing time and can identify delayed fracture healing in a subject.

In one embodiment, stimulating bone and/or fracture healing comprises converting cartilage to bone faster and/or improving quality of bone and/or forming better bone structure.

In one embodiment, accelerating bone and/or fracture healing comprises converting cartilage to bone faster.

In one embodiment, improving bone and/or fracture healing comprises improving quality of bone and/or forming better bone structure.

In another aspect, the disclosure provides a method for treating a subject having a bone fracture, comprising administering to the subject a composition according to the disclosure. In another embodiment, bone formation is increased in the fracture.

A clinician can assess need for bone healing and/or regeneration using known methods. In certain embodiments, the clinician uses experienced judgement, reduction in patient-reported pain, increased stiffness/mobility of the fracture, and a “hazy” appearance in the X-ray to estimate when the soft callus phase is peaking, for administration of a composition according to the disclosure.

β-Catenin mRNA

β-catenin is a multifunctional protein that plays a central role in physiological homeostasis (Shang, et al. 2017 Oncotarget 8(20):33972-33989). β-catenin is a pivotal component of the Wnt signaling pathway and is tightly regulated at the levels of protein stability, subcellular localization, and transcriptional activity. Indeed, Wnt is the chief regulator of β-catenin, regulating both the β-catenin-dependent (canonical Wnt) and -independent (non-canonical Wnt) signaling pathways.

Synthetic β-catenin mRNA provides a template for the synthesis of β-catenin protein, protein fragment, or peptide and provides a versatile delivery system for the β-catenin coding information to induce the production of β-catenin peptides and proteins in cells.

Disclosed herein is a non-destructible β-catenin gene that results in activation of the canonical Wnt pathway. This β-cat^{GOF} construct is generated through i) the deletion of exon 3 from the wild-type β-catenin, producing a ˜3.2 kb sequence (Harada, et al. 1999 EMBO J 18:5931-5942). Exon 3 contains the phosphorylation sites that cause proteasomal degradation of β-catenin by the destruction complex. Deletion of exon 3 therefore leads to transcription of the downstream Wnt effectors by preventing phosphorylation-mediated degradation of β-catenin.

In another embodiment, all uridine residues of the β-catenin^{GOF} mRNA are replaced with pseudouridines. In another embodiment, the pseudouridine is 1-methyl-3′-pseudouridine. In additional embodiments, the β-catenin^{GOF} mRNA is modified via mRNA capping, adding in untranslated regions (UTRs), and/or adding a polyA tail. The thus modified β-cat^{GOF} mRNA exhibits longer expression (thus higher Wnt signaling activation), less cytotoxicity/immunogenicity, enhanced stability, and/or increased transfection. The instant modification are to the linear mRNA.

SEQ ID NO: 1 provides the sequence of the full open reading frame of the non-degradable b-catenin lacking exon 3. SEQ ID NO:2 provides the sequence for which codon optimality was used to substitute some of the codons to improve stability. SEQ ID NO:3 provides the sequence of the protein that is encoded for by the mRNA and specifically shows that both SEQ ID NOS:1 and 2 lead to the same protein.

Additionally disclosed herein is β-cat^{GOF} mRNA that is engineered to be circular. circRNAs have several advantages over their linear counterparts. First, they are considerably more stable in vivo, as they lack 5′ and 3′ ends, which are the predominant targets of cellular RNases. This increases both the amount of—and the duration that—the encoded protein is expressed (Wesselhoeft, et al. 2019 Mol Cell 74:508-520). Second, as they lack 5′ ends, they don't require a 5′ cap for efficient translation. This is significant because trace amounts of uncapped mRNAs can induce immune responses. Thus, in one embodiment, β-cat^{GOF} protein is expressed from a circRNA. In specific embodiments, the circular mRNA would still have the above-iterated modified nucleosides (uridine replaced with pseudouridine), other potential codon optimizations/substitutions, and/or UTRs, but not the capping or polyA tail.

### Lipidic Transfecting Agents

mRNA had long been considered too unstable to be useful in pharmaceutical applications, due to its susceptibility to rapid degradation. However, mRNA can be optimized via modification to increase its intracellular stability, translational efficiency and uptake (Beck, et al. 2021 Mol Cancer 20:69).

A lipidic transfecting agent can be employed to stabilize, protect, and enhance delivery/uptake of β-catenin mRNA. For example, a lipid nanoparticle formulation can protect the β-catenin mRNA from extracellular RNases and improve its uptake in vivo. Lipid nanoparticles may include polymers, such as protamine, and/or cationic and ionizable lipids, with or without polyethylene glycol (PEG) derivatives, to enable complexing with the β-catenin mRNA via electrostatic interaction and condensing of the mRNA molecules (Zeng, et al. 2020 Curr Top Microbiol Immunol 10.1007/82_2020_217).

Lipids are amphiphilic molecules that contain three domains: a polar head group, a hydrophobic tail region and a linker between the two domains. Cationic lipids, ionizable lipids, and other types of lipid have been explored for mRNA delivery (Hou, et al. 2021 Nat Rev Mater 6(12): 1078-1094). Lipid nanoparticle-mRNA formulations typically contain lipid components other than cationic and ionizable lipids, such as phospholipids (for example, phosphatidylcholine and phosphatidylethanolamine), cholesterol or polyethylene glycol (PEG)-functionalized lipids (PEG-lipids), which can improve nanoparticle properties, such as particle stability, delivery efficacy, tolerability, and biodistribution. In specific embodiments, the β-catenin mRNA (for example, the β-catenin^{GOF} mRNA) is encapsulated in a lipid nanoparticle.

### Mineral Coated Microparticles (MCMs)

In addition to validating β-catenin mRNA (e.g., β-catenin^{GOF} mRNA) as a novel biologic for stimulating bone healing, a translationally relevant technology platform for local and controlled delivery is disclosed herein.

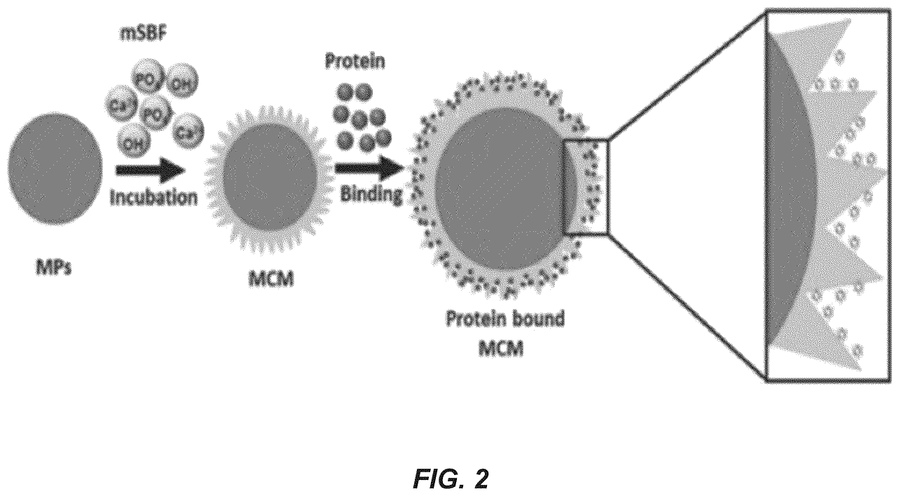

Mineral coated microparticles (MCMs) are disclosed herein as a therapeutic delivery platform. MCMs are 5-10μ diameter injectable biomimetic particles established for localized and sustained delivery of proteins, peptides, enzymes, and nucleic acids. MCMs are composed of a 5-8 μm resorbable β-tricalcium phosphate core with uniform calcium phosphate mineral coating. Calcium phosphate is deposited by incubation with modified simulated body fluids (mSBF) resulting in nucleation and growth of a nanometer-scale flaky mineral coating that offers a high surface area for binding and stabilizing biologics (Schmidt-Schultz and Schultz 2005 Biol Chem 386:767-776) (FIG. 2). Scanning electron microscopy of MCM demonstrates how mineral deposition creates bioinspired morphology with high surface area (not shown). The binding and release of biologics from MCM can be readily modulated by the physicochemical properties of the mineral coating. In specific embodiments, the physiochemical composition of MCMs is modified through the addition of fluoride or strontium (“fluoride- or strontium-doped”) to (1) activate Wnt signaling and (2) enhance therapeutic delivery of mRNA complexes to the fracture site. In another embodiment, the MCM can be doped with magnesium. In still another embodiment, the MCM can be doped with more than one of fluoride, strontium, and magnesium.

Thus, the disclosure additionally contemplates the administration of mineral coated microparticles (i.e., without the β-catenin mRNA complex) to a subject to activate the Wnt signaling pathway, to stimulate bone healing, to accelerate bone healing, to improve bone healing, to accelerate fracture repair, to treat malunion, and/or to stimulate bone regeneration in a subject.

In certain embodiments, the MCM are biocompatible and/or biodegradable. As used herein, the term “biocompatible” implies compatibility with a living system or living tissue, e.g., an animal or animal tissue, e.g. a human or human tissue, not being toxic, injurious, or physiologically reactive and/or causing a harmful immunological reaction. As used herein, the term “biodegradable” implies capability of being broken down, especially into innocuous products, by a natural system or natural components thereof, for example, in an animal subject, for example, in a human subject.

MCM can have any 3-dimensional shape. In certain embodiments, the architecture of the MCM is selected to benefit from a high aspect ratio. For example, rods, rectangles, wires, and the like have a high aspect ratio. In additional embodiments, the MCM are designed to enable a non-surgical delivery technology with high clinical relevance. Due to their small size, they can be easily injected for percutaneous delivery locally, for example, to a fracture site, and should not interfere with the normal healing process. At the same time, the MCM are large enough that they do not enter the bloodstream and float away.

In certain embodiments, the β-catenin mRNA (for example, the β-catenin^{GOF} mRNA) is bound to MCM. Such binding is, in a further embodiment, via adsorption, including due to electrostatic interactions and the large surface area of the mineral “flakes”. In further embodiments, the MCM binding the β-catenin mRNA (for example, the β-catenin^{GOF} mRNA) stabilize the mRNA. Furthermore, the controlled release provided by the MCM may result in a requirement for less mRNA, as the latter is provided slowly and is not quickly degraded. Thus, in certain embodiments, less β-catenin mRNA is required to achieve its biological activity, when it is bound to the MCM than when it is administered as an unbound complex.

In certain embodiments, the MCM are frozen or lyophilized for storage stability after the β-catenin mRNA-LNP (complex with lipidic transfecting agent, e.g., lipid nanoparticle) is bound to the same. In other embodiments, the MCM are frozen or lyophilized for storage stability after the β-catenin mRNA is bound to the same. In still other embodiments, the MCM and the β-catenin mRNA complex are assembled/mixed in a point of care setting.

### Wnt Signaling and its Activation for Bone Healing and Bone Regeneration

The Wnt signaling pathway is an osteoinductive program categorized according to the β-catenin-dependent canonical pathway and the β-catenin-independent non-canonical pathways (including the planar cell polarity and Ca²⁺-mediated pathways) (Gammons and Bienz 2018 Curr Opin Cell Biol 51:42-49). While some evidence suggests that the non-canonical pathways may play a role in regulating osteogenesis (Chen, et al. 2007 PLOS med 4:e249), the canonical Wnt/β-catenin pathway is well established for its role promoting osteogenesis and intramembranous bone repair (Monroe, et al. 2012 Gene 492:1-18). Canonical Wnt signaling regulates the transcription of genes involved in cellular processes such as proliferation, differentiation, self-renewal, and survival through the function of the transcriptional co-activator β-catenin. When this pathway is inactive, β-catenin is bound by a multiprotein “destruction” complex, which phosphorylates β-catenin, targeting it for ubiquitination and ultimately proteasomal degradation (Stamos and Weis 2013 Cold Spring Harb Perspect Biol 5(1):a007898). However, when the pathway is activated by Wnt ligand binding to the Frizzled and LRP5/6 co-receptors, the destruction complex is disrupted, enabling β-catenin to accumulate within the cytoplasm and translocate to the nucleus and activate transcription of target genes.

Mutating β-catenin in a way that it can no longer be phosphorylated prevents ubiquitination and degradation, resulting in activation of the Wnt signaling pathway. Chemical dopants such as fluoride or strontium can also disrupt the destruction complex, allowing for activation of the Wnt signaling pathway.

In comparison, little work has been done to determine the role of canonical Wnt signaling during endochondral bone formation and repair (Wong, et al. 2018 Front Bioeng Biotechnol 6:58). Chondrocytes become osteoblasts during endochondral bone development and repair. Furthermore, the canonical Wnt pathway likely acts as the key “molecular switch” required for the chondrocyte to osteoblast fate change. Thus, the Wnt pathway may play a significant, perhaps even critical, role in both intramembranous and endochondral bone repair, and its transient activation is achieved by the methods and compositions according to the disclosure.

Activation of the Wnt signaling through delivery of the modified/GOF β-catenin mRNA disclosed herein is indicated to be more safe than other therapeutic strategies to activate the Wnt pathway because of the known transience of intracellular mRNA expression, precluding the Wnt pathway from being “on” permanently. This is significant, because accumulation of β-catenin in the nucleus may promote the transcription of oncogenes such as c-Myc and CyclinD-1, which, if “on” permanently, could result in carcinogenesis and/or tumor progression of cancers including colon cancer, hepatocellular carcinoma, pancreatic cancer, lung cancer, and ovarian cancer (Shang, et al. 2017 Oncotarget 8(20):33972-33989).

Wnt-Activating mRNA Complex

To synergize the Wnt-activating capacity of fluoride- or strontium-doped MCM, the Wnt pathway can be directly activated by utilizing the MCM to deliver a stabilized β-catenin mRNA. A novel “gain of function” (GOF) β-catenin sequence is disclosed herein, adapted from a transgenic mouse in which the β-catenin lacks the phosphorylation sites that enable proteolytic degradation (Harada, et al. 1999 EMBO J 18:5931-5942). Transgenic expression of this sequence effectively promotes fracture repair in mice (Wong, et al. 2020 bioRxiv 2020.2003.2011.986141). Non-viral delivery of mRNA is a clinically viable approach that has recently shown high safety and efficacy in the COVID19 vaccine, as it avoids traditional, viral based delivery of genetic material leading to enhanced safety profiles, no risk of insertional mutagenesis, and no requirement of nuclear localization for efficacy. Delivering β-cat^{GOF} mRNA could circumvent the need to deliver Wnt ligands to activate the pathway and could produce a direct, cell-autonomous activation only within locally transfected cells. Traditionally, mRNA therapies are transient (hour time scale), which can be problematic when attempting to activate a pathway long-term, or permanently, but it is ideal for boosting a transient process that is part of the endogenous repair cycle—such as Wnt signaling during fracture repair. Novel, clinically relevant and translatable strategies to activate canonical Wnt pathway thus have tremendous therapeutic potential.

There have been few pioneering studies aimed at developing mRNA for orthopaedic applications. Bone regeneration studies involving BMP transfected by loading the mRNA onto various biomaterial platforms showed promising results in forming new bone, but the delivery platforms still required surgical implantation, and there was limited investigation into in vivo immunogenicity and efficacy. Thus, an injectable mRNA therapeutic could mitigate the need for additional surgeries and allow for optimization of the therapeutic delivery window.

### Administration

One aspect of the present disclosure includes administering a composition comprising β-catenin mRNA to a subject. Further aspects of the present disclosure include administering a composition comprising β-catenin mRNA to a subject. Still further aspects of the present disclosure include administering a composition comprising β-catenin mRNA encapsulated in a lipidic transfecting agent to a subject. Still further aspects of the present disclosure include administering a composition comprising β-catenin mRNA or β-catenin mRNA-LNP bound to mineral coated microparticles to a subject. In practicing the methods and uses according to certain embodiments of the disclosure, a composition according to the disclosure is administered to a subject.

In certain embodiments, a composition according to the disclosure is administered locally. The terms “local” and “locally”, as used herein, refer (in the context of a fracture) to in or adjacent to the bone defect, fracture gap, adjacent to the fracture site, adjacent to the fracture callus, along the periosteum, and/or within the intramedullary canal. In further embodiments, the composition may be administered to a tissue of a subject, at, next to, or near the fracture callus. The terms “local” or “locally” can also refer to where bone healing and/or regeneration is desired. By “locally” is meant directly to or directly adjacent to the site in which bone healing and/or bone regeneration is desired. In still another embodiment, the composition is injected into a bone defect of the subject.

Any convenient mode of administration may be employed. Modes of administration may include, but are not limited to injection (e.g., percutaneously, subcutaneously, intravenously, or intramuscularly, intrathecally).

In certain embodiments, the β-catenin mRNA, lipid transfecting agent, and/or MCM localize at the target location over a predetermined period of time. The term “localizes” is used herein in its conventional sense to refer to concentrating or accumulating administered β-catenin mRNA, lipid transfecting agent, and/or MCM, for example, within a predetermined area of the target site, such as within an area of 50 mm² or less, such as 40 mm² or less, such as 30 mm² or less, such as 25 mm² or less, such as 20 mm² or less, such as 15 mm² or less, such as 10 mm² or less, such as 9 mm² or less, such as 8 mm² or less, such as 7 mm² or less, such as 6 mm² or less, such as 5 mm² or less, such as 4 mm² or less, such as 3 mm² or less, such as 2 mm² or less, such as 1 mm² or less, such as 0.5 mm² or less, such as 0.1 mm² or less, such as 0.05 mm² or less and including a predetermined area of 0.001 mm² or less. In some instances, 10% or more of the administered β-catenin mRNA, lipid transfecting agent, and/or MCM in the composition localize within an area of the target site, such as 25% or more, such as 50% or more, such as 55% or more, such as 60% or more, such as 65% or more, such as 70% or more, such as such as 75% or more, such as 80% or more, such as 85% or more, such as 90% or more, such as 95% or more, such as 96% or more, such as 97% or more, such as 98% or more, such as 99% or more and including 99.9% or more of the administered β-catenin mRNA, lipid transfecting agent, and/or MCM in the composition localize within an area of the target site, such as within an area of 50 mm² or less, such as 40 mm² or less, such as 30 mm² or less, such as 25 mm² or less, such as 20 mm² or less, such as 15 mm² or less, such as 10 mm² or less, such as 9 mm² or less, such as 8 mm² or less, such as 7 mm² or less, such as 6 mm² or less, such as 5 mm² or less, such as 4 mm² or less, such as 3 mm² or less, such as 2 mm² or less, such as 1 mm² or less, such as 0.5 mm² or less, such as 0.1 mm² or less, such as 0.05 mm² or less and including a predetermined area of 0.001 mm² or less.

### Compositions

The disclosure provides compositions (pharmaceutical compositions) comprising β-catenin mRNA (e.g., β-catenin^{GOF} mRNA), a lipidic transfecting agent, and/or mineral coated microparticles for use in stimulating bone healing in a subject, accelerating bone healing in a subject, and/or improving bone healing in a subject. The disclosure also provides compositions comprising β-catenin mRNA (e.g., β-catenin^{GOF} mRNA), a lipidic transfecting agent, and/or mineral coated microparticles for use in accelerating fracture repair in a subject. The disclosure also provides compositions (pharmaceutical compositions) comprising β-catenin mRNA (e.g., β-catenin^{GOF} mRNA), a lipidic transfecting agent, and/or mineral coated microparticles for use in treating malunion in a subject. The disclosure provides compositions (pharmaceutical compositions) comprising β-catenin mRNA (e.g., β-catenin^{GOF} mRNA), a lipidic transfecting agent, and/or mineral coated microparticles for use in stimulating bone regeneration is stimulated in a subject. The disclosure also provides compositions (pharmaceutical compositions) comprising β-catenin mRNA (e.g., β-catenin^{GOF} mRNA), a lipidic transfecting agent, and/or mineral coated microparticles for use in activating Wnt signaling in a subject.

Compositions in accordance with the disclosure can be administered with suitable excipients, and/or other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell, et al., “Compendium of excipients for parenteral formulations”, PDA (1998), J Pharm Sci Technol 52:238-311.

In certain embodiments, the excipient is simply water, and in one embodiment, pharmaceutical grade water. In other embodiments, the excipient is a buffer, and in one embodiment, the buffer is pharmaceutically acceptable. Buffers may also include, without limitation, saline, glycine, histidine, glutamate, succinate, phosphate, acetate, aspartate, or combinations of any two or more buffers.

In other embodiments, a biodegradable matrix or scaffold is included in the composition. In further embodiments, the MCM are entrapped on the biodegradable matric or scaffold. In additional embodiments, the matrix is viscous, yet still flowable, and in other embodiments, the matrix is solid, semi-solid, gelatinous or of any density in between. Accordingly, in various embodiments and without limitation, the matrix is collagen, gelatin, gluten, elastin, albumin, chitin, hyaluronic acid, cellulose, dextran, pectin, heparin, agarose, fibrin, alginate, carboxymethylcellulose, Matrigel™ (a hydrogel formed by a solubilized basement membrane preparation extracted from the Engelbreth-Holm-Swarm (EHS) mouse sarcoma), hydrogel organogel, or mixtures and/or combinations thereof. Again, the worker of ordinary skill in the art will appreciate that any pharmaceutical grade matrix is amenable for use in a composition of the disclosure.

The amount of β-catenin mRNA can depend on the site of application, the condition being treated and the type of bioactivity desired and whether the mRNA is being administered on its own, encapsulated in a lipidic transfecting agent, and/or bound to a mineral coated microparticle.

In one embodiment, MCM are administered to a murine subject at a concentration of about 0.5 mg/kg to about 50 mg/kg. In another embodiment, this concentration range translates to a human equivalent dose range of about 0.04 mg/kg to about 4 mg/kg MCM administered (to a human subject). In still another embodiment, the human dose of MCM is standardized to about 3 mg to about 300 mg MCM based on an average human size of 75 kg.

In one embodiment, β-catenin (for example, β-catenin^{GOF}) mRNA is bound to the MCM at a concentration of 0.1 mg/1 mg to about 1 mg mRNA/1 mg MCM, which results in about 0.05 mg/kg to about 50 mg/kg mRNA delivered to a murine subject. In another embodiment, this range translates to a human equivalent dose range of about 0.004 mg/kg to about 4 mg/kg mRNA. In still another embodiment, the human dose is standardized to about 0.3 mg to about 300 mg mRNA based on an average human size of 75 kg.

In some embodiment, release of the β-catenin mRNA by the MCM is sustained release. By “sustained release” is meant that the mRNA is associated with the MCM to provide for constant and continuous delivery of the mRNA over the entire time the MCM are maintained in contact with the site of administration, such as over the course of 1 minute or longer, 5 minutes or longer, 10 minutes or longer, 15 minutes or longer, 30 minutes or longer, 45 minutes or longer, 1 hour or longer, 6 hours or longer, 12 hours or longer, 1 day or longer, 2 days or longer, 4 days or longer, 6 days or longer, 8 days or longer, 10 days or longer, 12 days or longer, 14 days or longer, 16 days or longer, 18 days or longer, or 20 days or longer.

In other embodiments, the MCM are degradable over time and deliver the β-catenin mRNA after a certain amount of the MCM has degraded. For example, an amount of the β-catenin mRNA may be delivered after every 10% of the MCM has degraded, such as after every 15% of the MCM has degraded, such as after every 20% of the MCM has degraded, such as after every 25% of the MCM has degraded, such as after every 30% of the MCM has degraded and including after every 33% of the MCM has degraded at the site of administration.

In still other embodiments, individual MCM employed in the present disclosure release a significant amount of the β-catenin mRNA immediately upon administration at the target site, such as for example 50% or more, such as 60% or more, such as 70% or more and including 90% or more of the β-catenin mRNA is released immediately upon administration. Thus, burst release kinetics are exhibited in certain embodiments. In yet other embodiments, the MCM release the β-catenin mRNA at a predetermined rate, such as at a substantially zero-order release rate, such as at a substantially first-order release rate or at a substantially second-order release rate.

In one embodiment, the MCM are associated with a targeting molecule that interacts with a target cell or tissue expressing a binding partner for said targeting molecule. In specific embodiments, the targeting molecule is selected from, without limitation, a cell adhesion molecule, a cell adhesion molecule ligand, an antibody immunospecific for an epitope expressed on the surface of a target cell type, and any member of a binding pair, wherein one member of the binding pair is expressed on the target cell or tissue of interest. In another embodiment, the electrostatic charge of the MCM is optimized to attract to cartilage, a highly negative matrix.

In certain embodiments, the dose of β-catenin mRNA/β-catenin mRNA bound to MCM may vary depending upon the age and the size of a subject to be administered, the type/severity of fracture, the location of fracture, conditions, route of administration, and the like. When the β-catenin mRNA/β-catenin mRNA bound to MCM disclosed herein are used for treating a bone fracture in a patient, it is advantageous to administer the β-catenin mRNA/β-catenin mRNA bound to MCM normally at a single dose of about 0.1 to about 100 mg/kg body weight. In specific embodiments, the dose/dosage is based on average release of β-catenin mRNA from MCM at the site of administration/the target site.

In certain embodiments, the frequency and the duration of the treatment (administration) can be adjusted. In certain embodiments, the β-catenin mRNA/β-catenin mRNA bound to MCM disclosed herein can be administered as an initial dose, followed by administration of a second or a plurality of subsequent doses of the β-catenin mRNA/β-catenin mRNA bound to MCM in an amount that can be approximately the same or less than that of the initial dose, wherein the subsequent doses are separated by at least one week, at least 2 weeks; at least 3 weeks; at least one month; or longer, based on a lack of adequate progression of healing parameters. In certain embodiments, a lack of adequate progression of healing parameters comprises no mineralization on X-ray, low mineralization on X-ray, no reduction in pain, minimal reduction in pain, no increase in stability, and/or minimal increase in stability. A clinician would be able to change the frequency and duration of treatment on a per patient basis based on their diagnosis and unique condition.

A composition of the present disclosure can, in certain embodiments, be delivered subcutaneously or percutaneously with a standard needle and syringe. In addition, a pen delivery device readily has applications in delivering a composition of the present disclosure. Such a pen delivery device can be reusable or disposable. A reusable pen delivery device generally utilizes a replaceable cartridge that contains a composition. Once all of the composition within the cartridge has been administered, and the cartridge is empty, the empty cartridge can readily be discarded and replaced with a new cartridge that contains the composition. The pen delivery device can then be reused. In a disposable pen delivery device, there is no replaceable cartridge. Rather, the disposable pen delivery device comes prefilled with the composition held in a reservoir within the device. Once the reservoir is emptied of the composition, the entire device is discarded.

### Therapeutic Uses

The β-catenin mRNA (e.g., β-catenin mRNA), β-catenin mRNA-lipidic transfecting agent, mineral coated microparticles (MCM), β-catenin mRNA-MCM, and β-catenin mRNA-lipidic transfecting agent-MCM according to the disclosure is/are each, in specific embodiments, useful for the treatment of bone defect or fracture, for the stimulation of bone healing, for the acceleration of bone healing, for the improvement of bone healing, for the treatment of malunion, delayed union, or non-union, for the stimulation of bone regeneration, and/or for the activation of Wnt signaling in a subject in need thereof (wherein a subject in need thereof may suffer from a condition or disorder or disease associated with bone defect, bone fracture, and the like). In additional embodiments, the β-catenin mRNA (e.g., β-catenin mRNA), β-catenin mRNA-lipidic transfecting agent, mineral coated microparticles (MCM), β-catenin mRNA-MCM, and β-catenin mRNA-lipidic transfecting agent-MCM according to the disclosure is/are each useful for the treatment of osteonecrosis or localized osteopenia.

In additional embodiments of the disclosure, the β-catenin mRNA (e.g., β-catenin mRNA), β-catenin mRNA-lipidic transfecting agent, mineral coated microparticles (MCM), β-catenin mRNA-MCM, and β-catenin mRNA-lipidic transfecting agent-MCM according to the disclosure is/are each, in specific embodiments, used for the preparation of a pharmaceutical composition or medicament for the treatment of bone defect or fracture, for the stimulation of bone healing, for the acceleration of bone healing, for the improvement of bone healing, for the treatment of malunion, delayed union, or non-union, for the stimulation of bone regeneration, and/or for the activation of Wnt signaling. In further embodiments, the β-catenin mRNA (e.g., β-catenin mRNA), β-catenin mRNA-lipidic transfecting agent, mineral coated microparticles (MCM), β-catenin mRNA-MCM, and β-catenin mRNA-lipidic transfecting agent-MCM according to the disclosure is/are each used for the preparation of a pharmaceutical composition or medicament for the treatment of osteonecrosis or localized osteopenia. In still another embodiment of the disclosure, the β-catenin mRNA (e.g., β-catenin mRNA), β-catenin mRNA-lipidic transfecting agent, mineral coated microparticles (MCM), β-catenin mRNA-MCM, and β-catenin mRNA-lipidic transfecting agent-MCM according to the disclosure is/are each used as adjunct therapy with another agent or another therapy known to those skilled in the art useful for the treatment of bone fracture, for the stimulation of bone healing, for the acceleration of bone healing, for the improvement of bone healing, for the treatment of malunion, for the stimulation of bone regeneration, and/or for the activation of Wnt signaling.

### Combination Therapies

In some embodiments of the methods and compositions according to the disclosure, an additional therapy or therapeutic agent is administered to the subject. In further embodiments, the additional therapy or therapeutic agent is a known therapy or agent used for bone healing, fracture repair/treatment, bone regeneration, and/or Wnt signaling activation.

In some embodiments, the additional therapy or therapeutic agent includes, but not limited to, protein supplements (e.g., including lysine, arginine, proline, glycine, cysteine, glutamine), antioxidants (e.g., vitamin E, vitamin C, lycopene, alpha-lipoic acid), mineral supplements (e.g., calcium, iron, potassium, zinc, copper, phosphorus, bioactive silicon), vitamin supplements (e.g., B (B6), C, D, and/or K), herbal supplements (e.g., comfrey, arnica, horsetail grass, Cissus quadrangularis), anti-inflammatory nutrients (e.g., quercetin, flavonoids, omega-3 fatty acids, proteolytic enzymes), and exercise. In other embodiments, the additional therapy or therapeutic agent is a Wnt signaling-activating agent including, but not limited to, R-spondin, Norrin, and Wnt protein. In still other embodiments, the additional therapy or therapeutic agent is a pro-chondrogenic (e.g., TGFb or maybe even PTH/PTHrP) drug.

In one embodiment, the additional therapy/therapeutic agent is administered in combination with a composition according to the disclosure. As used herein, the term “in combination with” means that at least one additional therapeutic agent/therapy may be administered prior to, concurrent with, or after the administration of a composition according to the disclosure. The term “in combination with” also includes sequential or concomitant administration of a composition according to the disclosure and at least one additional therapeutic agent/therapy.

In another embodiment, the additional therapy/therapeutic agent is administered concurrent with a composition according to the disclosure. “Concurrent” administration, for purposes of the present disclosure, includes, e.g., administration of a composition according to the disclosure and at least one additional therapeutic agent/therapy to a subject in a single dosage form, or in separate dosage forms administered to the subject within about 30 minutes or less of each other. If administered in separate dosage forms, each dosage form may be administered via the same route (e.g., both the composition according to the disclosure and the at least one additional therapeutic agent/therapy may be administered percutaneously, etc.); alternatively, each dosage form may be administered via a different route (e.g., the composition according to the disclosure may be administered percutaneously, and the at least one additional therapeutically active component may be administered orally). In any event, administering the components in a single dosage from, in separate dosage forms by the same route, or in separate dosage forms by different routes are all considered “concurrent administration,” for purposes of the present disclosure. For purposes of the present disclosure, administration of a composition according to the disclosure “prior to”, “concurrent with,” or “after” (as those terms are defined herein above) administration of at least one additional therapeutic agent/therapy is considered administration of a composition according to the disclosure “in combination with” at least one additional therapeutic agent/therapy.

### Kits

In an additional aspect, the disclosure provides kits, wherein the kits include at least one or more, e.g., a plurality of, the components needed to prepare a composition comprising β-catenin mRNA (for example, β-catenin^{GOF} mRNA), a lipidic transfecting agent, and/or mineral-coated microparticle, as disclosed herein. In certain embodiments, one or more of each component may be provided as a packaged kit, such as in individual containers (e.g., pouches). Kits may further include other components for practicing the subject methods, such as measuring and application devices (e.g., syringes), as well as containers for solutions such as beakers and volumetric flasks. In one embodiment, a kit may include a sterile vial and a needle to aspirate from vial prior to an injection. In another embodiment, a kit may include a lyophilized product or 2 lyophilized vials that are mixed together before injection. In still another embodiment, a kit may include a dual barrel syringe, wherein one side contains a lyophilized product and the other a mixing fluid/gel to be mixed together; the mixing may take place in the syringe or in the needle.

In addition, kits may include step-by-step instructions for how to practice the subject methods. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc. In yet other embodiments, the actual instructions are not present in the kit, but means for obtaining the instructions from a remote source, e.g., via the internet, are provided.

### EXAMPLES

The following examples are put forth so as to provide those of ordinary skill in the art with a complete disclosure and description of how to make and use the methods and compositions of the disclosure, and are not intended to limit the scope of what the inventors regard as their disclosure. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, room temperature is about 25° C., and pressure is at or near atmospheric.

### Example 1: Tuning the Mineral Composition of Bioinspired Microparticles for Osteogenic Activation and Enhanced mRNA Delivery to the Fracture Callus

In order to modify the chemical composition of existing mineral coated microparticles (MCMs) as a bimodal biomaterial platform to activate the Wnt pathway and enhance mRNA delivery, the addition of fluoride or strontium is tested for its potential to stimulate osteogenesis (Pan, et al. 2014 Toxicol Lett 225:34-42; Fromigue, et al. 2010 J Biol Chem 285:25251-25258). How these chemical dopants change mRNA transfection kinetics is also evaluated, as this is an aspect of mRNA delivery that needs to be improved. Currently, transfection kinetics remain on the order of hours to perhaps 1-2 days when using cationic lipid vehicles for mRNA delivery alone. Thus, MCM delivery of a reporter mRNA construct is tested and optimized in vitro and in vivo, with the goal of increasing mRNA transfection efficiency, prolonging mRNA expression, and stimulating Wnt pathway activation through mRNA-independent chemical modifications to the MCM. The MCM system is thus optimized for in vivo fracture repair. Inclusion of fluoride or strontium in the mineral composition of MCMs stimulates osteogenesis through activation of the Wnt pathway and prolongs the expression of mRNA in the fracture site.

### Mineral Coated Microparticles (MCM) as an Injectable Biomimetic Platform

The MCM platform is optimized for canonical Wnt pathway activation and delivery of mRNA. In certain embodiments, capabilities of the MCM platform essential to the success of the proposed approach include one or more of the following: (1) the ability to generate adaptable mineral coatings on the surface of resorbable β-tricalcium phosphate (β-TCP) microparticles, (2) the ability of MCMs to bind therapeutic biologics (e.g., growth factors, enzymes, mRNAs) and release them in a sustained fashion, and/or (3) the ability to locally release biologics in a temporally controlled manner. First, the methodology for successfully creating mineral coatings on β-TCP microparticles was validated to uniformly cover the entire surface with a nanoporous layer that dissolves slowly over time in aqueous solutions (FIG. 2). Second, efficient binding of proteins to MCMs with sustained release profiles indicates broad applicability of the mineral coatings to proteins, regardless of their electrostatic characteristics (Orth, et al. 2017 Eur Cell Mater 33:1-12; Dang, et al. 2016 Stem cells transl med 5:206-217; Orth, et al. 2019 J orthop res 37:821-831). Nucleic acid complexes bind to mineral coatings with high efficiency (over 70%) (Choi and Murphy 2010 Acta Biomater 6:3426-3435). Third, protein release was achieved in a site-specific manner over an extended time frame. Given that the properties of the mineral coatings (e.g., porosity, morphology, chemical composition, dissolution rate) are dictated by the conditions used for coating growth, it is demonstrated that biologics delivery can be systematically modulated by adding chemical dopants into the coating growth solution (Choi, et al. 2013 Sci Reports 3:1567).

Chemical Doping of MCMs for mRNA Independent Wnt Pathway Activation

The inclusion of chemical dopants in biomaterials to enhance bone formation has the advantage of overcoming stability issues inherent in biologics delivery, but less toxicity and fewer side-effects than a systemic administration of the dopants, since they are co-localized within the biomaterial (Marx, et al. 2020 Bone Rep 12:100273). Fluoride and strontium are focused on herein, based on their activation of Wnt.

Fluoride has been successfully incorporated into the mineral coating of MCM to demonstrate slowed mineral dissolution, changed coating morphology, and prolonged release of calcium and BMP2 (Yu, et al. 2014 Adv Func Mater 24:3082-3093) (data not shown). Fluoride-doped MCMs have also been shown to stabilize mRNA delivery in vitro (Fontana, et al. 2019 Mol Ther Nucl Acids 18:455-464), but fluoride has not been tested in vivo for mRNA therapy. Furthermore, activation of the Wnt pathway by fluoride-doped MCMs has not been tested to date. Fluoride activates the Wnt signaling pathway by inhibiting Wnt antagonists such as sclerostin, GSK-3β, and Dkk-1. In fact, it was shown that cells exposed to fluoride had higher accumulation of β-catenin and increased expression of osteogenic markers Runx2, alkaline phosphatase, collagen I, and osteonectin (Pan, et al. 2014 Toxicol Lett 225:34-42). Fluoride incorporation into the mineral coating could stabilize mRNA for prolonged delivery kinetics and activate the Wnt pathway in a mRNA-independent manner.

Strontium is also added herein as a chemical dopant. As with fluoride, strontium has been shown to activate the Wnt pathway to simultaneously increase bone formation and decrease bone resorption (Buehler, et al. 2001 Bone 29:176-179). Furthermore, strontium-enriched biomaterials consistently perform better than soluble strontium in vitro and in vivo in terms of bioactivity, cell proliferation, bone healing and osseo-integration (Marx, et al. 2020 Bone Rep 12:100273). Thus, in addition to testing the inclusion of fluoride, strontium doping within the mineral layer is tested at a concentration of 0.5-50 mM to each 50 mL of SBF. In vitro testing is done as for the fluoride-doped MCM.

β-TCP microparticles incubated in modified simulated body fluid (mSBF) with 4.2 mM bicarbonate (HCO₃⁻) for 7 days were used as a baseline MCM system herein (Yu, et al. 2014 Adv Func Mater 24:3082-3093). Fluoride is added to this baseline system at three different concentrations by incorporating 1, 10, or 100 mM of sodium fluoride (NaF) to 50 mL of mSBF (Yu, et al. 2014 Adv Func Mater 24:3082-3093). Strontium is also tested at three different concentrations (0.5, 5, or 50 mM) added to the mSBF. MCMs are synthesized using ACS grade reagents acceptable for food and medical use and are sterilized for 16 hours at 180° C. to destroy RNAses and remove eventual endotoxins. At the conclusion of coating formation, biomineral coatings are analyzed morphologically and compositionally using scanning electron microscopy (SEM), energy dispersive X-ray spectroscopy (EDS), X-ray diffraction (XRD), and Fourier transform infrared spectroscopy (FT-IR) as previously detailed in published work (Lee, et al. 2011 Adv Mater 23:4279-4284).

Osteogenic capacity of the MCMs is first tested in vitro using bone marrow-derived human mesenchymal stromal cells (hMSCs) and the chondrogenic cell line ATDC5, since these are the primary cell types within the fracture callus during the first phases of healing. Cells are cultured in 12-well tissue culture plates in standard basal medium at 20,000 cells/well. 12.5-250 μg MCM are added to each well and cultured for 3 to 48 hours. Metabolic health of the cells are then non-destructively analyzed with Presto Blue before harvesting for mRNA isolation using standard TriZOL protocols. Osteogenic genes (osteopontin, osteocalcin, alkaline phosphatase), downstream Wnt pathway genes (axin2, ctnb1) 104, and apoptotic gene (caspase3) are analyzed. Hypertrophic chondrocytes were used for all in vitro testing.

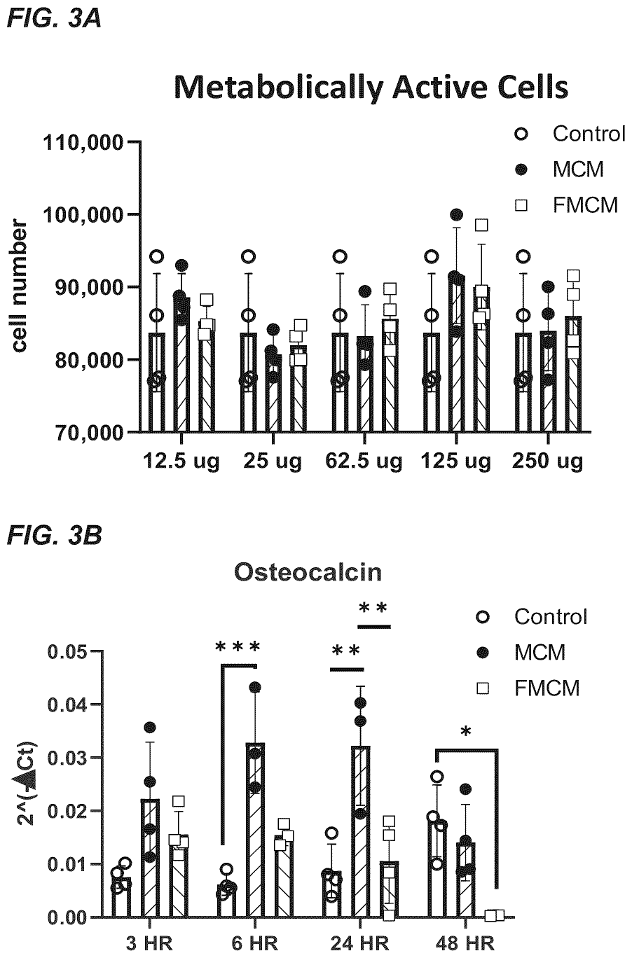

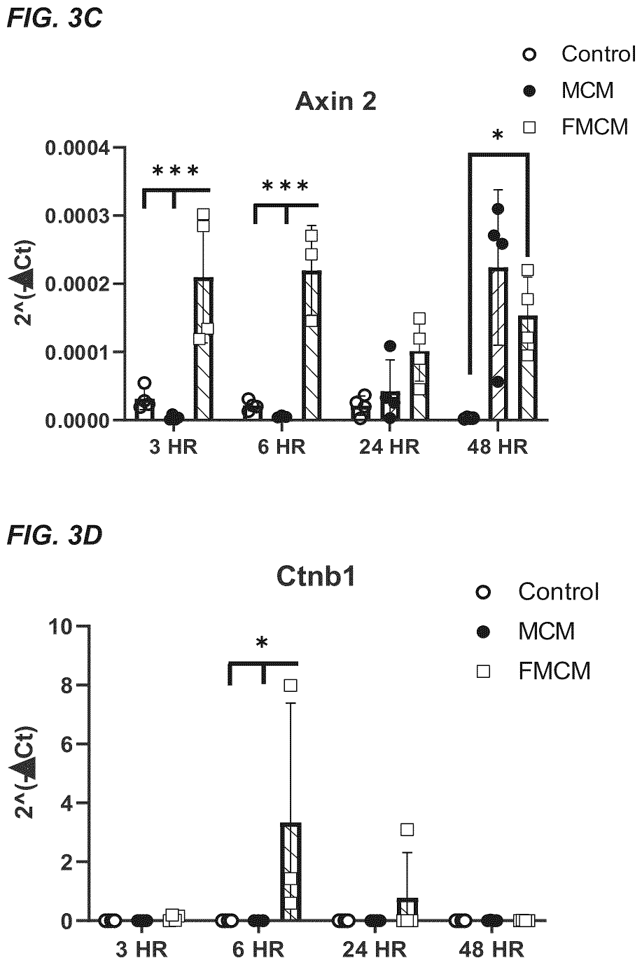

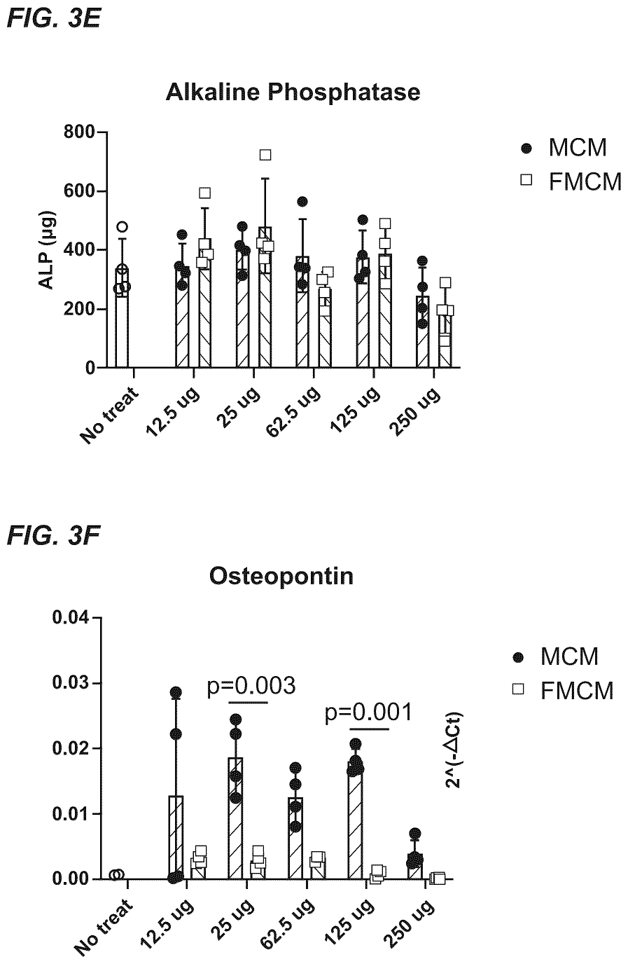

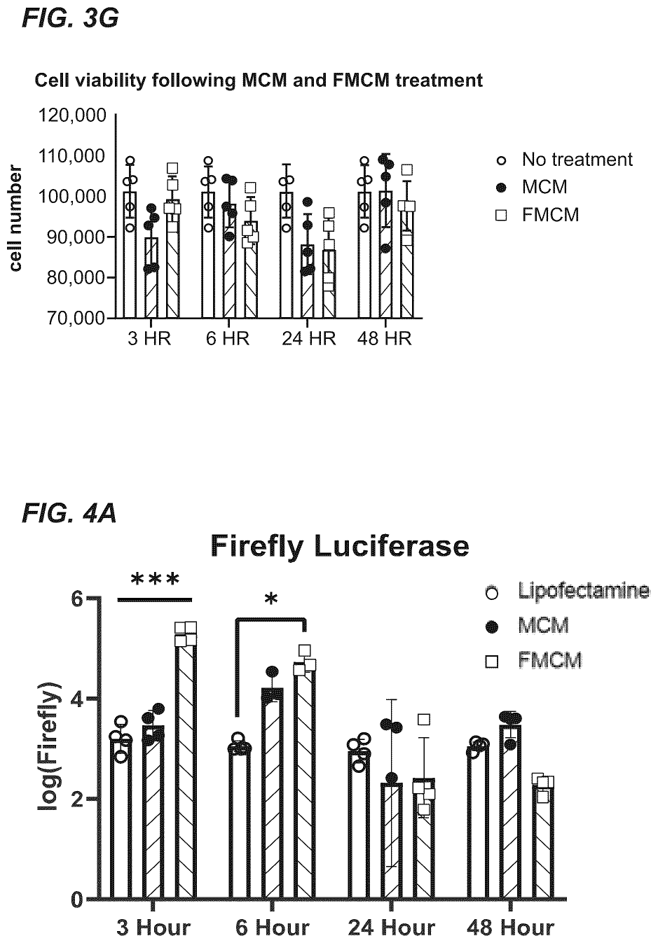

The fluoride (FMCM) and strontium (SMCM) doping are compared to baseline MCM, no-MCM (negative control), and standard osteogenic media (positive control) for cell proliferation and capabilities to promote osteogenesis. For added rigor, Wnt activation is quantified using the TOPFlash reporter system transfected into ATDC5s. TOPFlash comprises a vector containing TCF/LEF binding sites, the FOPFlash vector with mutated TCF/LEF sites (negative control), and the constitutively activated Renilla luciferase vector to correct for transfection efficiency through normalization. All in vitro testing is done with a minimum of 4-6 replicates. When comparing across multiple groups, an ANOVA is run to determine if there are statistical differences followed by Tukey's HSD post-hoc testing. Osteogenic characterization was determined by using qRT-PCR for various osteogenic markers (osteopontin (Opn), osteocalcin (Ocn), and downstream canonical Wnt marker, Axin2). Preliminary experiments indicate that MCM concentrations between 12.5-250 μg did not cause adverse cytotoxicity to ATDC5 cells (FIG. 3A), but can significantly upregulate osteocalcin (FIG. 3B) and the Wnt pathway markers (FIGS. 3C, 3D) in a time-dependent fashion. Indeed, no significant differences were found in number of cells between treatments (FIG. 3A) nor in level of secreted alkaline phosphatase treatment (FIG. 3E). Furthermore, MCM was found to have significantly more Ocn expression at every concentration tested compared to FMCM (FIG. 3B) and significantly more Opn expression at 25 and 125 μg than FMCM (FIG. 3F), yet FMCM had more Axin2 expression at all time points (FIG. 3C). Finally, cell viability was measured following MCM and FMCM treatment (FIG. 3G).

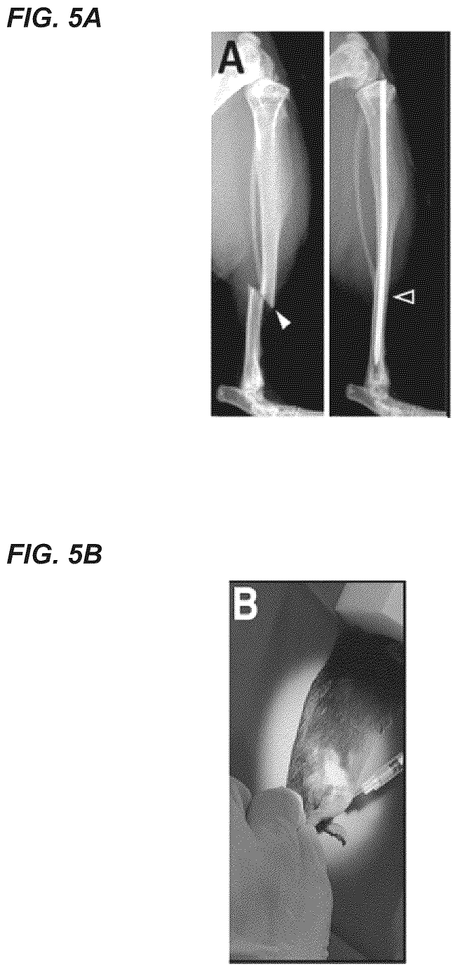

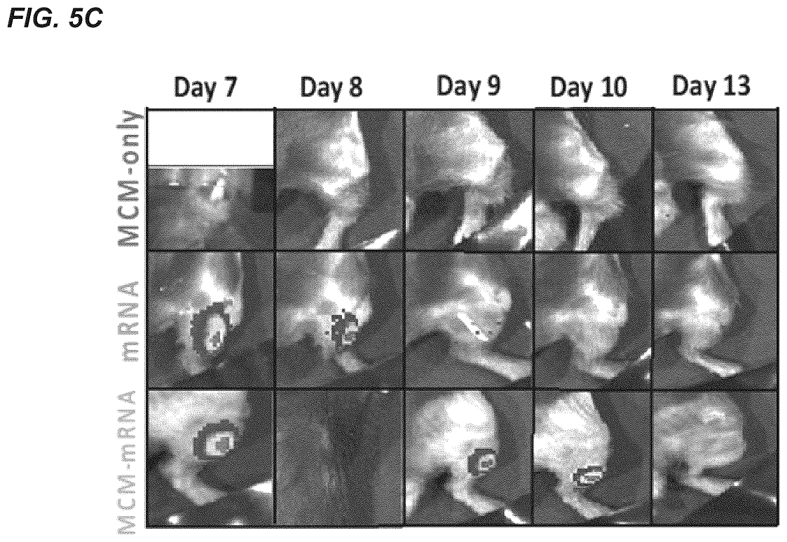

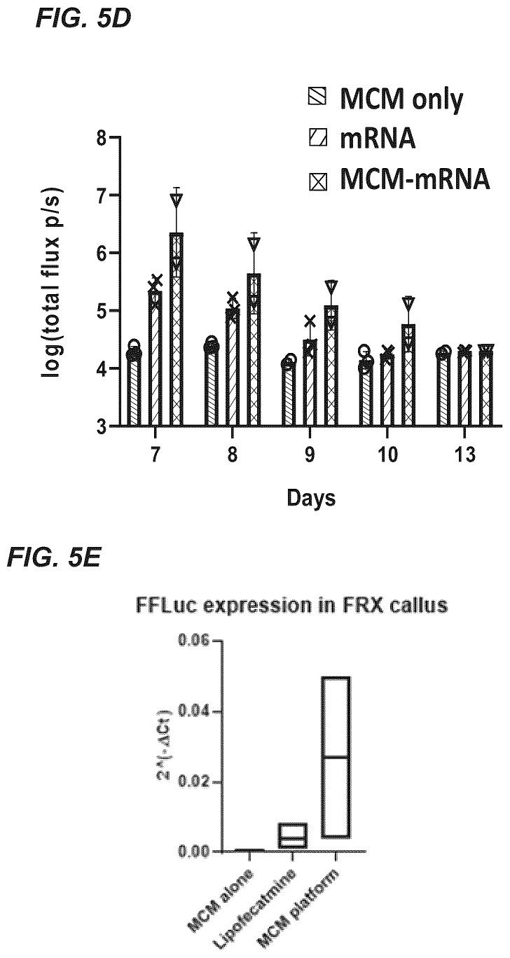

To further validate the technology, a clinically relevant murine fracture model is utilized to ensure a tissue specific response in the complex (whole animal) setting of repair. Murine surgeries are carried out to examine fracture healing outcomes in a tibia fracture model, because the tibia is one of the most commonly fractured bones, with a higher rate of delayed healing due its distal location and direct role in weight bearing (Praemer, et al. 1992 Musculoskeletal conditions US, 1^{st} edtn). Currently, the majority of tibia fractures are fixed clinically using an intramedullary nail to provide relative stability. As such, a pin-stabilized mid-shaft tibia fracture (FIG. 5A) is used herein.

Based on the in vitro results, the two MCM compositions that show the strongest activation of the Wnt pathway are tested in vivo compared to a placebo (negative control). MCMs are injected 6 days post fracture at two different concentrations to test their impact on intramembranous versus endochondral repair (Rivera, et al. 2020 Sci Rep 10:22241). The early regenerative and inflammatory response of the MCM is quantified 3 days after drug delivery as previously (Morioka, et al. 2019 Sci Rep 9:12199). Fracture calluses are dissected from the tibia and surrounding muscle to quantify the local regenerative and inflammatory responses. mRNA is extracted using TriZOL, cDNA is reverse transcribed, and qRT-PCR is performed for downstream Wnt targets, chondrogenic, osteogenic, and pro-inflammatory (Tnfα, Il1β, Il6) markers using validated SYBR primers. Increased Wnt targets and bone markers are an indication of an osteoanabolic effect, while no significant change in the inflammatory markers indicates the MCM are not immunogenic. Peripheral blood, spleen and liver tissue are also harvested to determine if systemic inflammation is triggered by the MCM. Spleen/liver tissue is analyzed by qRT-PCR to inflammatory markers (Morioka, et al. 2019 Sci Rep 9:12199). Pro-inflammatory markers in the blood are analyzed by ELISA. Utilizing the mean and standard deviation from a previous data set (Working, et al. 2020 J orthopaed res:office pub Orthopaed Res Soc 24776), a power analysis was conducted using G*Power to determine that 3 mice/group are required to achieve power=0.8 and α=0.05. 5 mice/group ensure rigor and account for potential variation with MCM delivery. As a result, there are 5 mice/group, 3 groups (2 MCM compositions, 1 control), and 2 MCM concentrations, at a single endpoint (3-days post MCM delivery) for a total of 25 mice. ANOVA and Tukey's HSD post-hoc testing is used as previously.

Quantitative μCT and Histomorphometry are the Primary Outcome Measures to Quantify Functional Changes to Fracture Repair with MCM Delivery.

A decreased cartilage fraction and increase in bone fraction at day 14 post-fracture indicates improved fracture repair. Fractures are fixed in 4% PFA and μCT completed using our Scanco μCT80 scanner. Bone mineral density, bone volume, trabecular thickness, and trabecular density are calculated (Rivera, et al. 2021 bioRxiv doi.org/10.1101/2021.11.16.468864). Subsequently, legs are decalcified and embedded in paraffin. Serial sections (10 μm) are cut, and every 10th slide is stained with Hall Brunt's Quadruple (HBQ) to identify bone (red) and cartilage (blue). Tissue volume and fracture callus composition are quantified using standard principles of histomorphometry on blinded samples. In addition to capturing cartilage and bone fraction, fibrous tissue volume and marrow space are also quantified to comprehensively characterize the fracture callus composition.

Sex-related differences in fracture healing of adult mice have not previously been found when corrected for weight differences; thus, an equal mix of male and female mice are used. A power analysis was conducted in G*Power using the mean and standard deviation from published studies (Wong, et al. 2020 bioRxiv 2020.2003.2011.986141; Rivera, et al. 2020 Sci Rep 10:22241) to determine that 10 mice/group/time would be required for histomorphometry and μCT45 to achieve a power level >80% with an effect size d=1.5 and a significance level of 5%. Statistical comparison of multiple groups is performed via a one-way ANOVA (α=0.05). Tukey's HSD post-hoc analysis is performed on data sets with statistical difference by ANOVA to determine which groups differ statistically. Based on the same groups as above, this analysis requires 10 mice/group, 3 groups (2 MCM compositions, 1 control), and 2 MCM concentrations at a single endpoint (14-days post fracture) for a total of 50 mice. All mice tested are between 10-14 weeks old to avoid the age-related delay in fracture repair (Clark, et al. 2017 Curr osteopor rep 15:601-608).

Chemical Doping of MCMs to Prolong mRNA Delivery Kinetics and Reduce Cytotoxicity.

In addition to serving a Wnt activating function, MCMs can enhance mRNA delivery by improving intracellular transfection and significantly alleviating cytotoxicity of the cationic lipid vector in vitro (Fontana, et al. 2019 Mol Ther Nucl Acids 18:455-464). Specifically, MCM-mediated mRNA delivery was beneficial, because it gradually delivered the mRNA complexes, thereby mitigating their disruptive effect on the cell's membrane. MCMs also stimulated endosomal activity leading to increased mRNA internalization, likely due to the presence of locally increased concentrations Ca²⁺ and PO₄³⁻ dissolved from the mineral coating.

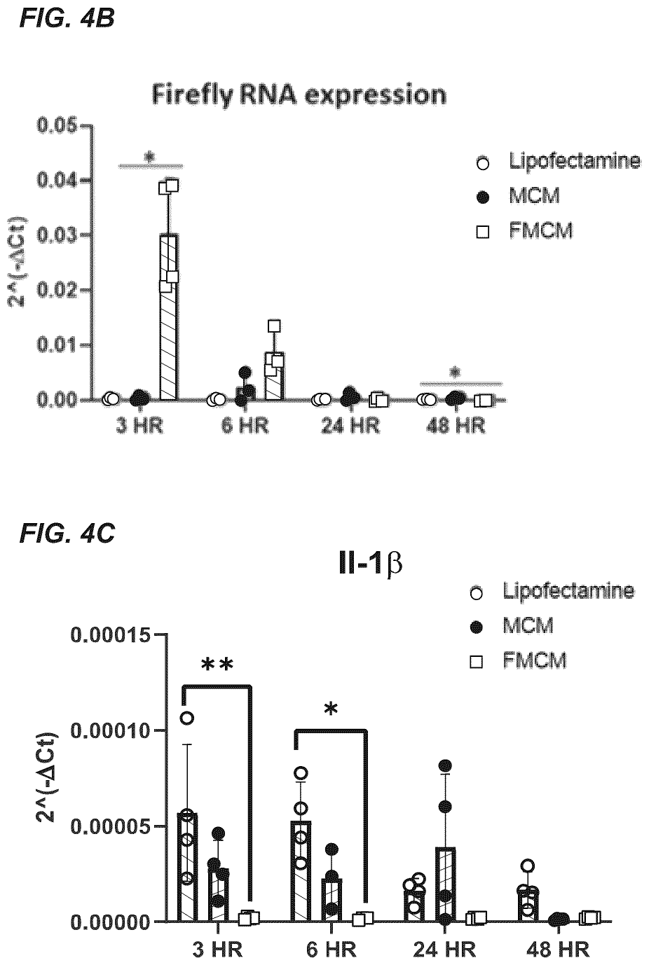

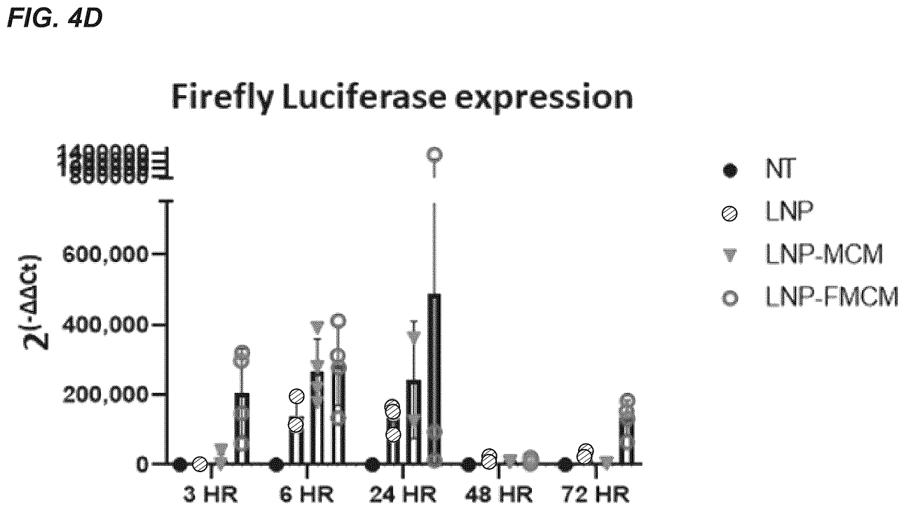

Transfection efficiency, or the magnitude of transfection, and transfection kinetics, or the duration of transfection, were assessed for each of the delivery platforms. Both MCM and FMCM require a lipid complex to carry and stabilize the mRNA, such as Lipofectamine™. To formulate the delivery platform, first the lipid vesicles form a complex with the nucleic acid. After adding the MCM, lipid-nucleic acid complexes physically interact with the mineral coating. Firefly luciferase (FLuc) mRNA was used as a reporter gene to quantify transfection using qRT-PCR. FMCM was found to significantly enhance transfection at 3 hours following treatment (FIG. 4B, p=0.019). Log transformed analysis of 2(−ΔCt) shows granular differences between delivery platforms (FIG. 4A). Preliminary data support that FMCM enhances the expression of firefly luciferase mRNA but significantly reduces expression of the pro-inflammatory IL1β in chondrocytes in vitro (FIG. 4C), as well as IL-4 in chondrocytes in vitro (data not shown). As immunogenicity is frequently associated with mRNA delivery, it was important to develop a gene delivery platform which minimizes an inflammatory response. Finally, when reporter mRNA were encapsulated in lipid nanoparticles (LNPs) and treated on chondrocyte cells (the cells of a fracture callus), time-dependent expression of the reporter mRNA from the chondrocytes was exhibited. For 20,000 cells/well in 12-well plates, the treatments consisted of LNP-mRNA complex at 0.25 μg mRNA/well, and MCM or FMCM at 25 μg/well. Both the MCM and fluoride doped MCM improved mRNA transfection with the lipid nanoparticles (LNPs) relative to LNP alone (FIG. 4D). Furthermore, the fluoride doped MCM (FMCM) resulted in the fastest transfection into the cells, the greatest magnitude of mRNA expression, with the longest expression.

A first in vivo study using the MCM to deliver mRNA to a pin-stabilized murine tibia fracture was run (FIG. 5A). Firefly luciferase mRNA (10 μg/mouse, Trilink Biotech cat #L-7202-100) was encapsulated into the standard commercial cationic lipid vector Lipofectomine™ (cat #LMRNA001) according to manufactures protocols and then incubated with 100 μg MCMs for 1 h at room temp on a shaker in OptiMEM. MCM alone, luciferase mRNA in Lipofectomine™ (Luc/mRNA/Lipo), or MCM-Luc/mRNA/Lipo were then percutaneously delivered to the fracture callus 6 days following surgery (FIG. 5B). Luciferase expression was measured longitudinally in vivo using IVIS to provide a semi-quantitative assessment of the magnitude and length of expression. (FIG. 5C) Luciferase expression within the fracture callus was also quantified at gene level. (FIG. 5D) Finally, to validate the IVIS imaging results, RNA was harvested from the fracture callus and was probed for firefly luciferase via qRT-PCR. MCM platform was shown to have more luciferase mRNA expression (FIG. 5E). As evident from the IVIS imaging and mRNA expression, Luciferase expression remained highly localized to the fracture region, and MCMs significantly prolonged the expression of Luciferase in the fracture callus. Thus, when used as a delivery carrier for complexed mRNA, MCMs can considerably alleviate the cytotoxicity of non-viral vectors, promote cellular internalization of mRNA complexes, improve transfection efficiency, and extend transfection kinetics.

Based on preliminary data, there is strong evidence that the MCM platform can significantly enhance and prolong delivery of the mRNA. MCM, FMCM, or SMCM are rigorously tested for their ability to improve the magnitude and kinetics of mRNA delivery on both in vitro (MSC, chondrocyte, e.g. FIGS. 4A, 4C) and in vivo (fracture callus, e.g. FIGS. 5A5D) relative to Lipofectamine and placebo controls using luciferase as a convenient reporter construct. The amount of MCMs is fixed based on the above results, and the mRNA concentration is changed from 0.1 μg mRNA/μg MCM to 1 μg mRNA/μg MCM. While no luciferase expression was observed outside of the leg in the preliminary studies (FIG. 5C), thorough biodistribution studies are conducted using IVIS and mRNA to check for systemic expression of luciferase in blood cells, spleen, liver, and lungs. Using robust immunohistochemistry protocols (Hu, et al. 2017 Dev 144:221-234; Wong, et al. 2020 bioRxiv 2020.2003.2011.986141; Wong, et al. 2020 J orthopaed res: office pub Orthopaed Res Soc doi: 10.1002/jor.24904). Which cells get transfected is defined. Activation of an innate inflammatory response is measured through a standard complete blood cell differential and by measuring pro-inflammatory genes in the fracture callus and spleen. Local macrophage (F480) and neutrophil (Ly6) infiltration into the fracture call is quantified from immunohistochemistry using histomorphometry (Clark, et al. 2020 Aging Cell 19:e13112). Apoptosis is evaluated by Caspase3 and TUNEL staining (Hu, et al. 2017 Dev 144:221-234). Inflammatory responses are both expected and necessary for effective fracture healing (Bahney, et al. 2019 J orthopaed res:offic pub Orthopaed Res Soc 37:35-50), as such, only outcome measures that increase apoptosis and inflammation by more than 25% relative to placebo are considered clinically meaningful and excluded. All in vitro testing is done with a minimum of 4-6 replicates. For the in vivo studies, a power analysis based on the preliminary data (FIGS. 5A-5D) indicates that 6 mice/group are required to achieve a power level >80% with an effect size d=1.5 and a significance level of 5%. Experimental design thus includes 6 mice/group, 5 groups (3 MCM compositions, Lipofectamine only, placebo control), 2 mRNA concentrations with two endpoints (3- and 7-days post-delivery) for a total of 108 mice. Murine studies are done on adult wild type mice with an equal number of male and female mice. When comparing across multiple groups, an ANOVA is run to determine if there are statistical differences followed by Tukey's HSD post-hoc testing. Sex-dependent responses are tested.

Thus, the instant example was designed to tailor the chemical properties of the MCM as an injectable delivery platform to activate Wnt signaling through mRNA-independent incorporation of mineral dopants and prolong the expression of mRNA with a decreased the host immune response. Based on the preliminary data (FIGS. 3A-3G), one, or both, of the chemical dopants (fluoride, strontium) are expected to activate the canonical Wnt pathway as a bioactive platform to promote osteogenesis. In specific embodiments, (1) significantly enhanced Wnt activation as measured by axin2/Cntb1 gene expression and TOPFlash activity relative to placebo, (2) increased osteogenic gene expression in vitro and in vivo, and (3) increased bone formation at day 14 in vivo with the MCM compared to placebo indicate efficacy. In specific embodiments, for the objective of improving mRNA delivery, (1) significantly enhanced luciferase expression as measured by IVIS and luciferase qRT-PCR, (2) prolonged luciferase expression, and (3) decreased inflammatory response relative to Lipofectamine delivery alone indicate efficacy. Percutaneous delivery of MCM to the fracture callus should produce localized expression of the mRNA with minimal ectopic effects. In the unlikely event that MCM leak outside the area of interest, the overall charge of the mineral coating can be changed to enhance electrostatic interactions with negatively charged chondrocytes in the fracture. This would increase the cell-MCM interactions and minimize the likelihood that MCM can move away from the area of interest. Alternatively, MCM could be co-injected with a polymeric carrier, such as alginate (Krebs, et al. 2010 J biomed mater res, Pt A 92:1131-1138), to secure them in place. Should the transfection kinetics not be ideal, intervention is possible at multiple levels. For example, if changing chemical properties of the mineral coating is not sufficient to enable sustained expression of mRNA, the β-TCP core material can be changed to have longer or slower degradation rate.

### Example 2. Optimization of a β-Catenin mRNA Lipid Nanoparticle Complex for Direct Activation of Canonical Wnt Signaling

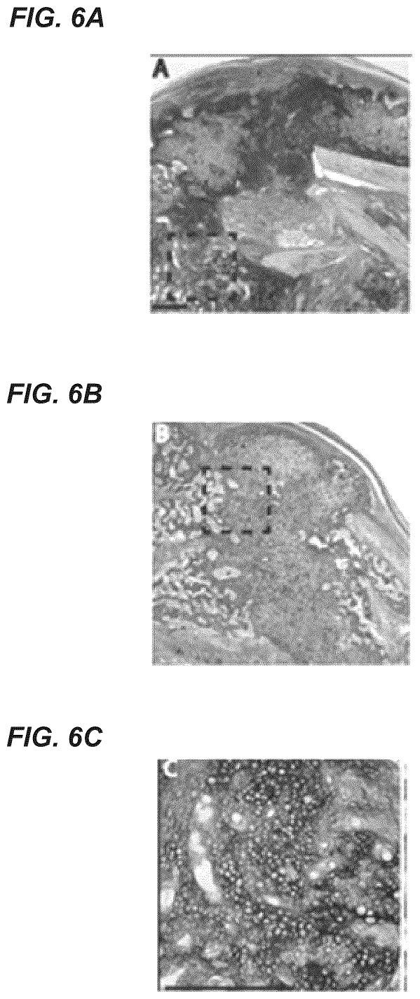

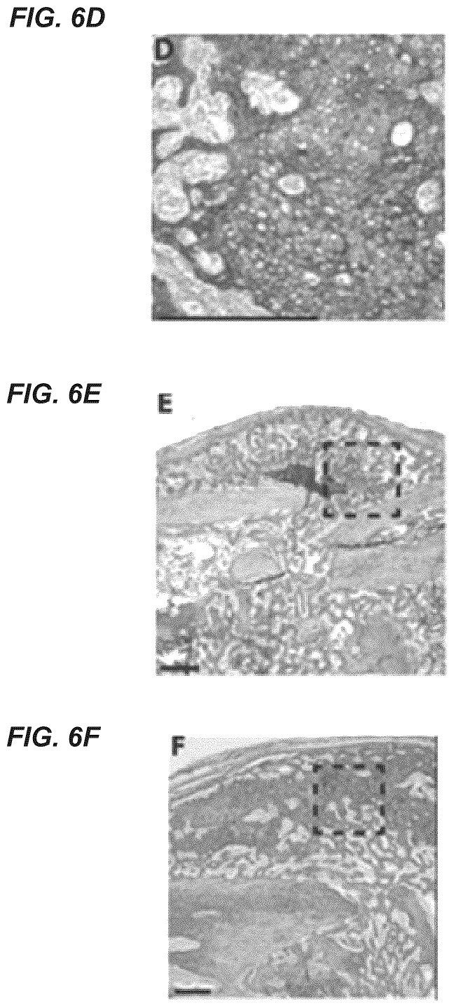

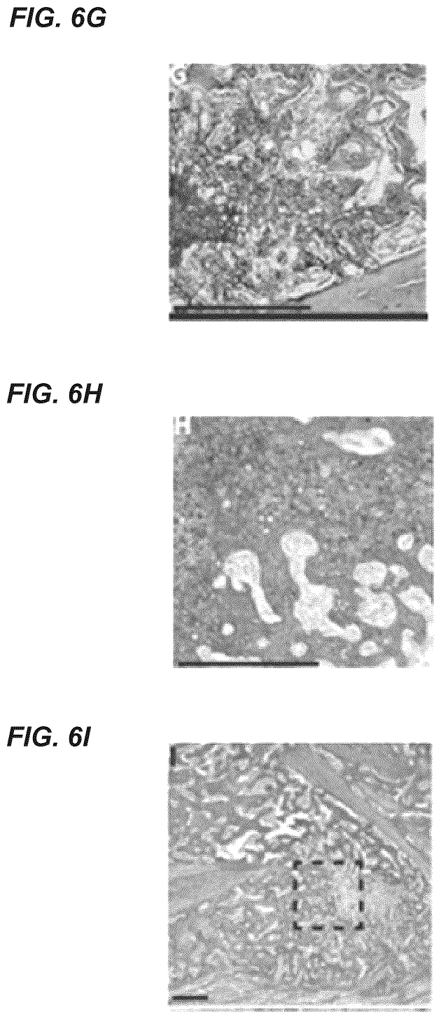

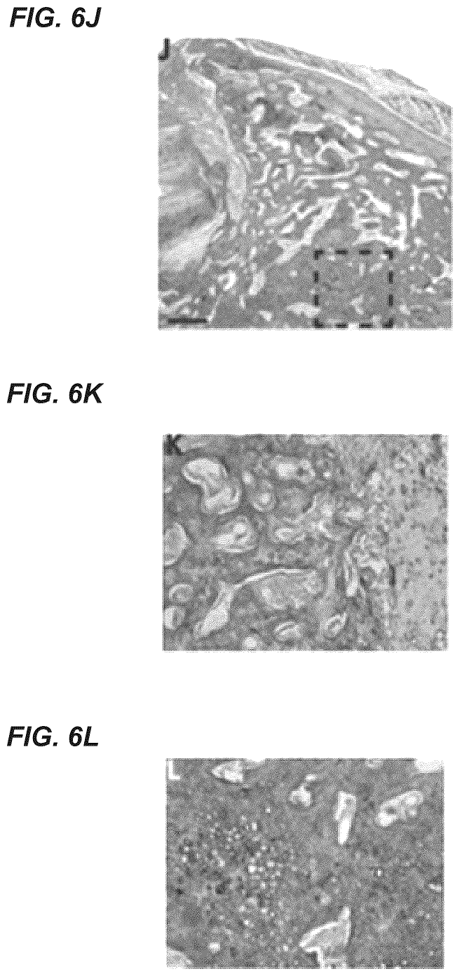

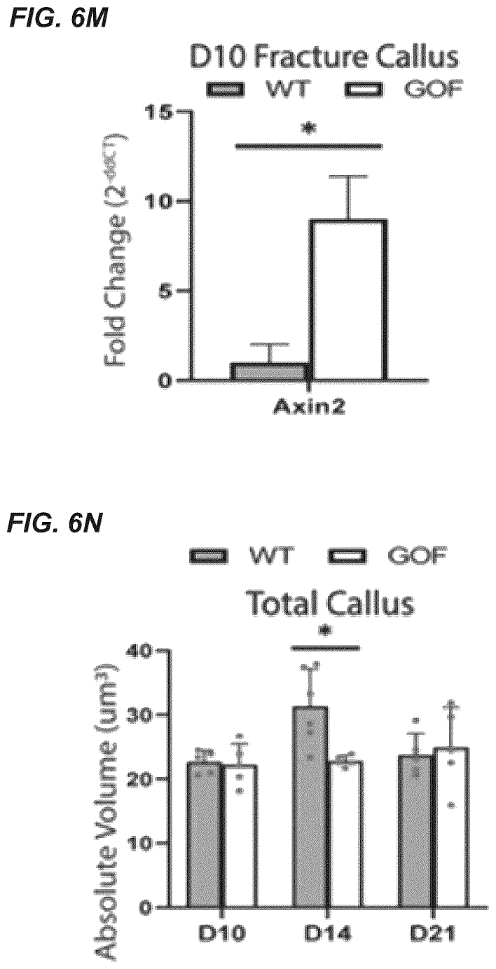

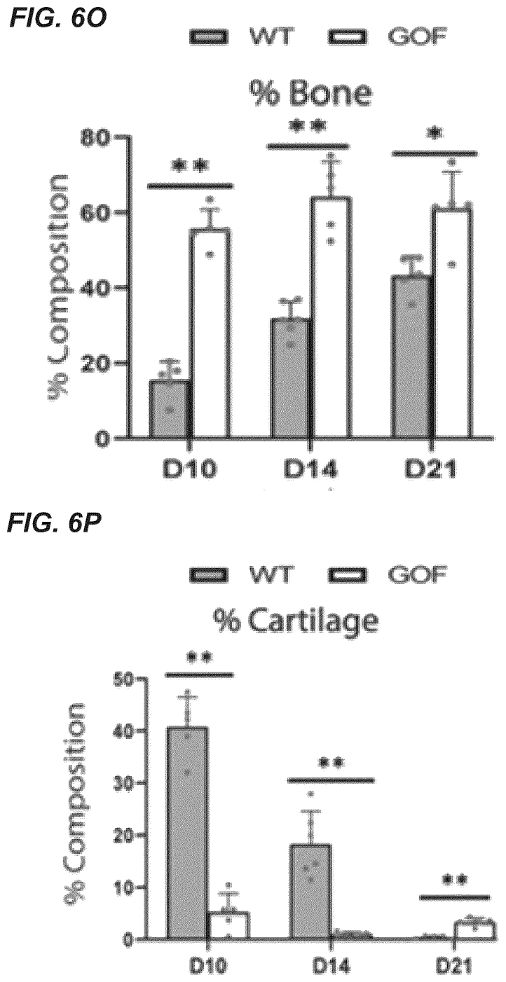

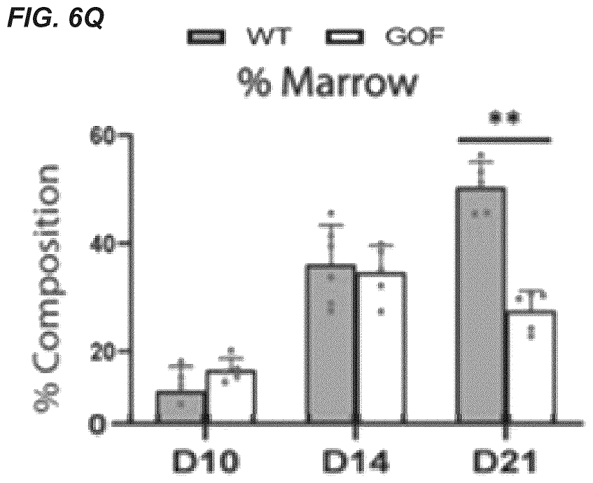

In order to engineer a novel β-cat^{GOF} mRNA complex that can be injected to directly activate canonical Wnt signaling in the fracture callus, the β-cat^{GOF} transgene that has been demonstrated to accelerate fracture repair when transiently induced in the fracture callus (FIGS. 6A-6Q) was translated into a mRNA therapeutic by adding modified nucleosides, optimized untranslated regions (UTRs), a poly(A) tail, and clean capping. The β-cat^{GOF} mRNA therapeutic is optimized by employing codon optimality to develop multiple sequences that can be functionally tested with the goal of increasing stability, improving translation, and decreasing immunogenicity. Next, the in vitro and in vivo efficacy of the optimized linear β-cat^{GOF} mRNA are compared to a novel circular β-cat^{GOF} mRNA (circRNA) construct to further improve mRNA expression kinetics and Wnt pathway activation, with reduced immunogenicity. The mRNA is initially delivered with the standard commercial reagent Lipofectamine™ as a non-optimized cationic lipid for transfection. Next, clinical grade engineered lipid nanoparticles are tested as delivery vectors for the mRNA with the goal of reducing Lipofectamine™-associated cytotoxicity. Because therapeutic mRNA and lipid nanoparticles are both known to have a tissue-specific response, they should be designed and tested in an application-specific manner. mRNA construct optimization (nucleoside modifications, codon optimality, circRNA) delivered within engineered lipid nanoparticles is likely to prolong intracellular expression, amplify Wnt pathway activation, and minimize the inflammatory response within the fracture callus.

β-Cat^{GOF} mRNA Therapeutic Construct to Activate Canonical Wnt Signaling

Canonical Wnt signaling plays an essential role in intramembranous ossification (Monroe, et al. 2012 Gene 492:1-18), and this pathway is also required for endochondral conversion of cartilage to bone (Houben, et al. 2016 Dev 143:3826-3838). Therapeutically the Wnt pathway is challenging to directly activate, because Wnt ligands are lipidated (Willert, et al. 2003 Nature 423:448-452). Consequently, existing Wnt therapies (such as the FDA-approved EVENITY®) deliver hydrophilic antibodies to Wnt inhibitors to indirectly activate the Wnt pathway (Canalis 2013 Nat Rev Endocrinol 9:575-583). While EVENITY® has proven anabolic in the treatment of osteoporosis, clinical studies testing efficacy in fracture repair have shown no benefit (Schemitsch, et al. 2020 J Bone Joint Surg, Amer vol 102:693-702), indicating that systemic delivery of monoclonal antibodies is insufficient to stimulate localized repair. Thus, mRNA technology could be useful in directly activating Wnt signaling to promote fracture repair.

Using transgenic mice, conditional expression of a non-destructible β-catenin transgene (β-cat^{GOF}) was shown to accelerate bone repair when transiently induced from days 6-10 post-fracture (FIGS. 6A-6Q). The β-cat^{GOF} construct is a ˜3.2 kb sequence generated through the deletion of exon 3 from the wild-type β-catenin (Harada, et al. 1999 EMBO J 18:5931-5942). Exon 3 contains the phosphorylation sites that cause proteasomal degradation of β-catenin by the destruction complex. Deletion of this exon then leads to transcription of the downstream Wnt effectors by preventing phosphorylation-mediated degradation of β-catenin (Stewart, et al. 2000 J Bone Miner Res 15:166-174). qRT-PCR analysis of the Wnt target gene axin2 confirmed that canonical Wnt signaling was over-activated by induction of the β-cat^{GOF} transgene (FIG. 6M). At a functional level, β-cat^{GOF} expression accelerated fracture repair, as evidenced by the increased bone and decreased cartilage fraction in the fracture callus at all timepoints compared to control (FIGS. 60 and 6P). Thus, histomorphometric quantification confirms increased bone and decreased cartilage composition in fracture callus. The images portray formation of new trabecular (woven) bone within the fracture callus surrounded by chondrocytes and hypertrophic chondrocytes.

This β-cat^{GOF} transgene sequence was translated into an mRNA therapeutic using an RNAcore. The β-cat^{GOF} mRNA therapeutic contains the deletion of exon 3 from the wild-type β-catenin (as in the transgene) but contains additional modifications: incorporation of untranslated regions (UTRs) known to confer both high translatability and stability, replacement of all uridine residues with 1-methyl-3′-pseudouridine, high efficiency mRNA clean capping, a poly(A) tail, and a nanoluciferase as a reporter element. Nucleoside modifications were part of the core design, as they have proven to be a key advance in reducing the immunogenicity and increasing the effectiveness of mRNA therapies (Krienke, et al. 2021 Science 371:145-153; Corbett, et al. 2020 NEJM 383:1544-1555). Collectively, the described mRNA modifications represent the baseline β-cat^{GOF} mRNA and serve as the platform to which sequence- and tissue-specific changes are added to improve functionality.

To engineer a β-cat^{GOF} mRNA construct with a therapeutically useful stability and translation profile, codon optimality is employed. Codon optimality in the RNA biology of eukaryotic systems (Presnyak, et al. 2015 Cell 160:1111-1124; Medina-Munoz, et al. 2021 Genome Biol 22:14), is distinct from codon optimization in bacterial systems, which only addresses changes that account for tRNA abundance. Recent research into codon optimality has shown that certain synonymous codons confer an additional degree of mRNA stability and/or more efficient translation than other codons for a particular amino acid (Presnyak, et al. 2015 Cell 160:1111-1124; Forrest, et al. 2020 PloS one 15:e0228730). Several guiding principles have been delineated for codon optimality: first, that codon optimality is tissue- and cell-type specific, and second, that codons enriched in guanosines and cytosines are more likely to be optimal than those lacking those nucleotides (Mauger, et al. 2019 PNAS USA 116:24075-24083). Minimizing the occurrence of uridines is a common practice for mRNA therapies, as it also reduces the immunogenic potential of the mRNA therapeutic. Several publicly available algorithms (e.g., icodon.org) can be employed to calculate codon optimality and generate three distinct mRNA sequences to evaluate in cell culture experiments. These three tested mRNA constructs all contain a nanoluciferase reporter element to label the mRNA and be encapsulated into Lipofectamine™ as a standard, non-optimized cationic lipid vector for transfection. The mRNA complex showing the longest cellular expression, highest level of Wnt pathway activation, and least inflammatory/cytotoxic response in vitro is selected as the baseline linear mRNA.

### Structural Enhancement Through Engineered Circular RNA

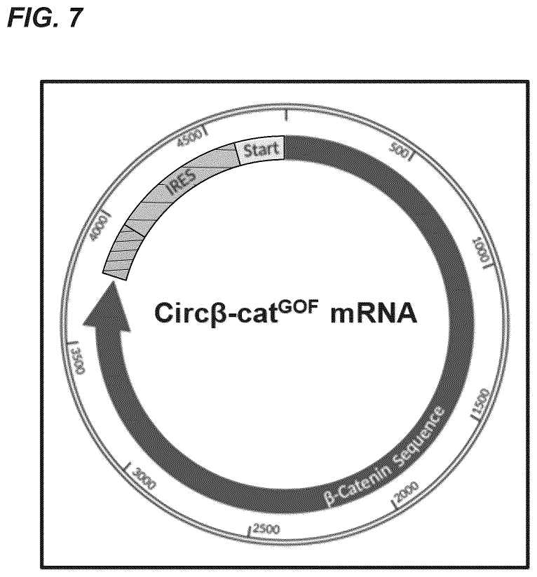

The RNAcore has the ability to generate circular RNAs (circRNAs) by expressing proteins from circular internal ribosome entry sites (IRES) (Wesselhoeft, et al. 2019 Mol Cell 74:508-520). circRNAs have several advantages over their linear counterparts. First, they are considerably more stable in vivo, as they lack 5′ and 3′ ends, which are the predominant targets of cellular RNases. This increases both the amount of, and duration that, the encoded protein is expressed. Second, as they lack 5′ ends, they don't require a 5′ cap for efficient translation. This is significant, as trace amounts of uncapped mRNAs can induce immune responses. This technology is applied to create a novel circβ-cat^{GOF} (FIG. 7) that is compared to both the baseline and optimized linear mRNA constructs, using cell culture experiments. The mRNA therapy that produces the longest cellular expression, highest level of Wnt pathway activation, and least inflammation is a candidate β-cat^{GOF} mRNA for in vivo validation.

In Vitro Validation of β-Cat^{GOF} mRNA Therapeutic

Transfection efficiency and kinetics of the therapeutic linear and circular β-cat^{GOF} are first tested in vitro in chondrocytes (ATDC5) and MSCs, since these are the primary cell types within the fracture callus. Transfection efficiency is tested by quantifying the percentage of cells that express the nanoluciferase (coded for in the β-cat^{GOF} mRNA construct), though qRT-PCR to quantify luciferase expression, and a luminometer. Luciferase expression is quantified starting at 2 hrs and continues until expression is no longer detectable.

Functional validation testing measures the magnitude and temporal sequence of canonical Wnt pathway activation following treatment with the β-cat^{GOF} mRNA complex using qRT-PCR to Wnt pathway targets (axin2, cntb1I) and the TOPFlash fluorescent reporter system. The β-cat^{GOF} mRNA complex is compared to Wnt3a ligand (50 ng/mL) (Hannousch, et al. 2008 PloS one 3:e3498) as a positive control, with scrambled mRNA, Lipofectamine alone, and placebo as negative controls. All in vitro testing is done with a minimum of 4-6 replicates. When comparing across multiple groups, an ANOVA is run, followed by Tukey's HSD post-hoc testing.

Since exogenous mRNA is known to stimulate a cell-specific innate immune response, the immune and apoptotic responses of the chondrocytes and MSCs treated with the various β-cat^{GOF} mRNAs are compared. Cytotoxicity is evaluated in vitro using the non-destructive PrestoBlue™ Cell Viability Assay and flow cytometry to quantify cellular apoptosis using RealTime-Glo™ Annexin 5. qRT-PCR is used to measure canonical pro-inflammatory genes (Tnfα, Il-1β, Il-6). The final β-cat^{GOF} mRNA is chosen based on the mRNA structure maximizing Wnt activation and producing the least inflammatory phenotype.

Preclinical Fracture Model Validation of mRNA Therapy

Following in vitro validation and selection of the final β-cat^{GOF} mRNA therapy, efficacy is validated in the target clinical application of fracture repair. The mRNA complex (10 μg mRNA) is injected locally to the fracture 6-days post-operatively. Transfection efficiency and kinetics are visualized within the fracture callus using daily live imaging on IVIS to provide a semi-quantitative assessment of the magnitude and length of expression of the nanoluciferase-β-cat^{GOF} mRNA complex (FIG. 5C). Analogous to the TOPFlash in vitro system, in vivo fluorescent transgenic Wnt reporter mice have been developed (Barolo 2006 Oncogene 25:7505-7511) and allow the visualization of the Wnt pathway activation daily using IVIS. Two Wnt reporter models are utilized: the Axin2-eGP mouse104 (Jackson 016998) (Rivera, et al. 2020 Sci Rep 10:22241), and the ins-TOPeGFP mouse with 6 LEF/TCF consensus binding sites and a minimal promoter derived from pTOFLASH129 (Jackson 013752). Quantitative assessments of transfection efficiency and pathway activation are done following imaging by isolating RNA from the fracture callus tissue and quantifying luciferase and axin2 expression using qRT-PCR. Finally, immunohistochemistry is performed (Bahney, et al. 2014 J Bone Miner Res 29:1269-1282) to determine which cells are transfected and activating the Wnt pathway. Expression is quantified daily for up to one week post-delivery, or until luciferase expression is lost. As with the in vitro testing, functionality of the β-cat^{GOF} mRNA complex in vivo is compared to Wnt3a ligand (25 mg/kg) as a positive control; scrambled mRNA, empty cationic lipids, and placebo injections are performed as negative controls (minimum of 5 mice/group). Statistical significance is tested using ANOVA followed by Tukey's HSD.

Immunogenicity, cytotoxicity, and biodistribution of the final β-cat^{GOF} mRNA are also evaluated 1, 3, and 5 days following therapeutic delivery. Inflammatory responses are expected and lend to effective fracture healing; as such, outcome measures that increase apoptosis and inflammation by more than 25% relative to placebo are considered clinically meaningful. These analyses are done on the same mice used to test functionality of the β-cat^{GOF} mRNA, above.

### Engineered Lipid Nanoparticles for Clinical Translation

Non-viral delivery of mRNA is used herein, because it offers an increased safety profile over viral delivery with no risk of insertional mutagenesis. However, non-viral mRNA transfection is highly inefficient without a cationic lipid vector. Commercially available lipid vectors, such as Lipofectomine™, increase the stability of mRNA and promote cellular internalization, but toxicity associated with these vectors prevents clinical translation. To develop a clinically translatable mRNA therapy, Lipofectomine™ is compared to clinical grade engineered lipid nanoparticles (LNP), with the goal of reducing cytotoxicity while maintaining good transfection efficiency. LNPs are synthesized using a benchtop NanoAssemblr™ to rapidly combine the organic and aqueous phases using microfluidic mixing to formulate nanoparticles in a reproducible manner. The organic phase is composed of lipids (DLin-MC3, DSPC, Cholesterol, DMG-PEG at the ratio, 50:10.5:38:1.5) in ethanol, while mRNA is dissolved in sodium acetate buffer (pH=4) as the aqueous phase. Synthesized LNPs are dialyzed overnight in 1×PBS and filtered using 0.22 μm filters for sterilization prior to characterization. These mRNA-LNP are roughly 60-80 nm in size and achieve 90% RNA encapsulation efficiency. When stored at 4° C., mRNA-LNPs are stable for at least 4 weeks, which mimics the stability of typical liposomal formulations currently on the market.

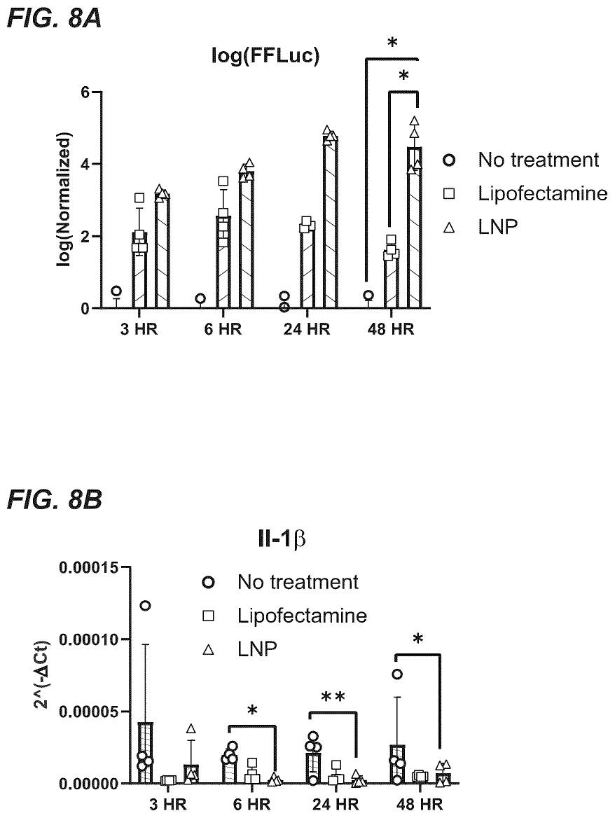

To test whether LNPs can achieve mRNA delivery comparable to Lipofectamine, the efficiency and kinetics of the mRNA expression are evaluated in ATDC5 and MCSs. Preliminary data suggest that the engineered LNPs lead to at least equivalent expression of luciferase compared to Lipofectamine (FIG. 8A). Cytotoxicity and inflammatory response of the lipid vectors are also evaluated to determine the relative cytotoxicity of engineered LNPs compared with Lipofectamine. Preliminary data further suggest that LNPs reduce pro-inflammatory IL1β response in chondrocytes (FIG. 8B) For added rigor, intracellular trafficking and internalization analyses were performed with the fluorescent LNPs. For these studies, cells are seeded into 96-well plates and imaged using a Nikon Fluorescent microscope on the Okolab Bioreactor to facilitate live cell imaging. Cells are subsequently fixed after 48 hours with 4% paraformaldehyde and stained with antibodies to localize the LNPs relative to the endosome (EEA1), lysosome (LAMP1) and nucleus (DAPI). Stained cells are analyzed using NIS elements and ImageJ. In specific embodiments, defining the mRNA-LNP concentrations that achieve cellular transfection equivalent to or better than Lipofectamine is desired. All in vitro testing is done with a minimum of 4-6 replicates. Groups will be compared using ANOVA followed by Tukey's HSD post-hoc.

Based on the genetic evidence that the β-cat^{GOF} transgene can accelerate fracture repair, this can likely be translated into an effective mRNA therapy. A circular β-cat^{GOF} mRNA therapy engineered with codon optimality is likely to produce maximal stability of the mRNA construct, enhanced translation and Wnt pathway activation, with the least amount of induced immunogenicity. The amount of β-cat^{GOF} mRNA therapy (10 μg) was initially chosen based on BMP mRNA bone regeneration studies, but if mRNA-driven Wnt activation is lower than through Wnt3a (25 mg/kg) delivery, a more extensive dose validation study can be completed. The engineered LNPs likely improve transfection efficiency and reduce cellular toxicity relative to Lipofectomine™ to produce a clinically translatable mRNA complex. Since the circRNA design is novel, one concern is that it may not be efficiently encapsulated in LNPs due to its chemical modifications and tertiary structure. This can reduce the efficacy of mRNA-LNP in vitro and in vivo. However, the optimized linear β-cat^{GOF} mRNA can be tested within the LNPs while adjusting the LNP formulation using different ratio of lipids. In specific embodiments, stimulating local Wnt activation with the mRNA technology that parallels Wnt3a-mediated protein activation without a clinically relevant increased immune reaction indicates efficacy.

### Example 3. Comparison of Therapeutic Efficacy of Wnt Activating Platforms in the Murine Model of Fracture Repair

The therapeutic efficacy of the combinatorial β-cat^{GOF}-MCM platform is tested in a murine fracture model in the context of alternative approaches to stimulate the Wnt pathway, specifically: MCM only, β-cat^{GOF} mRNA complexes only, localized Wnt3a injections, and systemic administration of the Wnt agonist EVENITY® (along with appropriate controls). An mRNA-based approach should solve the existing technology gap to directly activate canonical Wnt signaling, leading to the strongest activation of the Wnt pathway, and can synergize with the MCM platform to address previous limitations of mRNA therapies. Furthermore, testing is carried out to determine whether early (intramembranous) or late (endochondral) delivery of Wnt-activating therapies is more effective. Fracture healing and inflammatory response are rigorously quantified using standard techniques (gene expression, μCT, histomorphometry), as well as the collagen X fracture biomarker (Working, et al. 2020 J orthopaed res: office pub Orthopaed Res Soc doi: 10.1002/jor.24776) throughout the time course of repair. β-cat^{GOF}-MCM therapy is likely to effectively accelerate endochondral fracture healing.

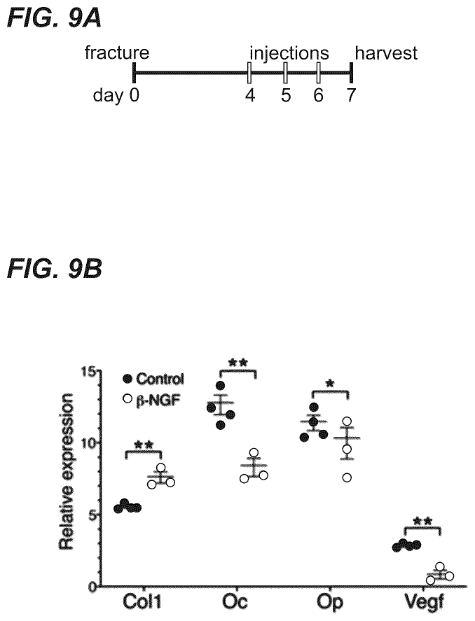

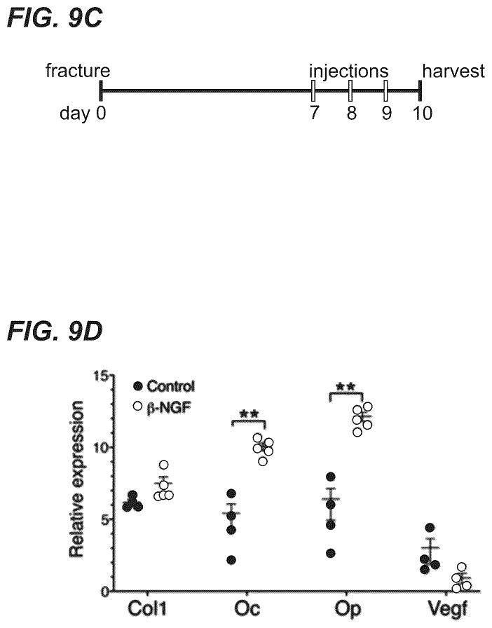

When testing therapies for fracture healing, most drugs are given immediately after fracture by default, and as such, target intramembranous bone formation. Described herein is an effort to improve efficacy of novel therapies by delivering them at a timing that corresponds to their endogenous expression in fracture healing (“developmental engineering”). The importance of timing in therapeutic delivery is demonstrated by showing that local injections of nerve growth factor (NGF) only upregulated osteogenesis when delivered later, to the endochondral phase of repair, correlating to endogenous NGF expression in the fracture callus (FIGS. 9A-9D). Since Wnt signaling is critical to both intramembranous and endochondral repair, it is unclear what the best timing is for Wnt activation. This is tested by delivering the Wnt activating therapeutics disclosed herein either 3- or 6-days after the fracture to target either intramembranous or endochondral repair, respectively. Delivery of the therapeutic at the time of fracture is clinically irrelevant, because endogenous regeneration requires the initial inflammatory phase to subside.

### In Vivo Validation of β-Cat^{GOF}-MCM Therapeutic Complexes

To maintain high clinical relevance, the β-cat^{GOF}-MCM complexes disclosed herein are validated using a preclinical pin-stabilized murine tibia fracture model and rigorous evaluation of healing. Murine stabilized tibia fractures are as described above and shown in FIG. 5A. Therapeutic injections are given using a Hamilton syringe under fluoroscopy as described above and shown in FIG. 5B at either 3- or 6-days post-fracture. The experimental groups include the following experimental and control groups: negative control, positive control (Wnt3a ligand), MCM only, mRNA complex (no MCM), mRNA-MCM, and pharmaceutical equivalency.

The early regenerative response and biodistribution of the therapy is quantified by qRT-PCR 3 days after drug delivery as detailed above. Briefly, this includes gene expression analysis of Wnt targets, along with standard chondrogenic, osteogenic, pro-inflammatory, and apoptotic markers using validated SYBR Green primers. Systemic inflammation is also evaluated using a CBC, while looking for off-target expression of Wnt expression in the spleen. Since extensive biodistribution is done on each of the components above, safety is compared across the platforms and the assays targeted based upon outcomes above. Utilizing the mean and standard deviation from previously published data set (Morioka, et al. 2019 Sci Reports 9:12199), a power analysis was conducted using G*Power to determine that 3 mice/time are required to achieve a power=0.8 and α=0.05. 5 mice x 6 groups (30 total) are planned to account for any additional variation associated with treatments. ANOVA and Tukey's HSD post-hoc testing are used to evaluate significance as previously.

As detailed above, functional changes in fracture repair are measured by quantitative μCT and histomorphometry as primary outcome measures. To quantify both the rate and extent of healing across groups, the animals are evaluated 10-, 14-, 21- and 28-days post fracture. Using a Scanco μCT80 scanner bone mineral density, bone volume, trabecular thickness, and trabecular density are calculated. Subsequently, tissue volume and fracture callus composition are quantified using standard principles of histomorphometry on blinded samples. In addition to capturing cartilage and bone fraction, fibrous tissue volume and marrow space are quantified, as previously, to comprehensively characterize fracture tissue. Power analysis and sample size justification result in 10 mice/group/time being required for μCT and histomorphometry. Given 6 groups and two drug delivery start time (3- or 6-days), a total of 180 mice are needed to complete this study. Statistical comparison of multiple groups are performed via ANOVA (α=0.05), followed by Tukey's HSD post-hoc analysis. All mice tested are between 10-14 weeks old to avoid age-related effects of fracture repair and tested on both sexes.

The use of a circulating collagen X (“Cxm”) biomarker to quantify the biological composition of the fracture callus adds to the quantification of fracture healing (Coghlan, et al. 2017 Sci Transl Med 9: doi: 10.1126/scitranslmed.aan4669). Collagen X is the canonical marker of chondrocyte hypertrophy and is transiently expressed as cartilage turns into bone (FIG. 1). Cxm levels have been correlated to gene and protein expression in fracture healing (Working, et al. 2020 J orthopaed res:office pub Orthopaed Res Soc doi: 10.1002/jor.24776). This serum bioassay is a novel, non-destructive, longitudinal measurement that allows the comparison of molecular signatures of repair in control vs therapeutically treated mice. Blood is collected from the tail vein (˜25 μl, non-destructive) 3 days prior to—and 14 days following fracture, and then via cardiac punch at terminal time point of the study.

The validation of the Wnt activating β-cat^{GOF}-MCM platform in fracture healing disclosed herein is carried out. An mRNA-based approach should solve the existing technology gap to directly activate canonical Wnt signaling, leading to the strongest activation of the Wnt pathway, and can synergize with the MCM platform to address previous limitations of mRNA therapies. In specific embodiments, efficacy is based on the clinical objective of developing a therapy that results in earlier bone formation.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

What is claimed is:

1. A method of stimulating bone healing, accelerating bone healing, and/or improving bone healing in a subject, comprising administering a composition comprising β-catenin mRNA complex to the subject.

2. The method of claim 1, wherein the bone healing is bone fracture healing.

3. The method of claim 1, wherein bone regeneration is stimulated in the subject.

4. The method of claim 3, wherein the regeneration is within a bone fracture site in the subject.

5. The method of claim 1, wherein the β-catenin mRNA has a gain-of-function mutation.

6. The method of claim 5, wherein one or more codons of the β-catenin^{GOF} mRNA are modified to: i) optimize stability and/or translatability of the mRNA; and/or ii) reduce immunogenicity of the mRNA.

7. The method of claim 1, wherein the β-catenin mRNA is circular.

8. The method of claim 1, wherein the β-catenin mRNA complex is encapsulated in a lipidic transfecting agent.

9. The method of claim 8, wherein the lipidic transfecting agent is a lipid nanoparticle.

10. The method of claim 1, wherein the β-catenin mRNA complex is bound to mineral coated microparticles (MCM).

11. The method of claim 10, wherein the MCM are spherical or rod-shaped.

12. The method of claim 10, wherein the MCM comprise a mineral coating comprising Ca²⁺ and/or PO₄³⁻.

13. The method of claim 10, wherein the MCM comprise a mineral coating comprising at least one chemical dopant.

14. The method of claim 13, wherein the at least one chemical dopant is fluoride or strontium.

15. The method of claim 1, wherein the composition further comprises an osteoconductive graft.

16. The method of claim 15, wherein the osteoconductive graft is selected from the group consisting of an autograft, an allograft, demineralized bone matrix, and a collagen scaffold.

17. The method of claim 1, wherein the composition is administered to the subject via injection.

18. The method of claim 17, wherein the injection is into a bone defect of the subject.

19. The method of claim 1, wherein the subject has a bone fracture, and the composition is administered during the intramembranous periostal repair phase or at the end of the endochondral repair phase of fracture healing.

20. The method of claim 1, wherein the subject has a bone fracture, and the composition is administered during the intramembranous periostal repair phase and at the end of the endochondral repair phase of fracture healing.

21. A composition comprising β-catenin mRNA complex.

22. The composition of claim 21, wherein the β-catenin mRNA has a gain-of-function mutation.

23. The composition of claim 22, wherein one or more codons of the β-catenin^{GOF} mRNA are modified to: i) optimize stability and/or translatability of the mRNA; and/or ii) reduce immunogenicity of the mRNA.

24. The composition of claim 21, wherein the β-catenin mRNA is circular.

25. The composition of claim 21, wherein the β-catenin mRNA complex is encapsulated in a lipidic transfecting agent.

26. The composition of claim 25, wherein the lipidic transfecting agent is a lipid nanoparticle.

27. The composition of claim 21, wherein the β-catenin mRNA complex is bound to mineral coated microparticles (MCM).

28. The composition of claim 27, wherein the MCM are spherical or rod-shaped.

29. The composition of claim 27, wherein the MCM comprise a mineral coating comprising Ca²⁺ and/or PO₄³⁻.

30. The composition of claim 27, wherein the MCM comprise a mineral coating comprising at least one chemical dopant.

31. The composition of claim 30, wherein the at least one chemical dopant is fluoride or strontium.

32. The composition of claim 21, wherein the composition further comprises an osteoconductive graft.

33. The composition of claim 32, wherein the osteoconductive graft is selected from the group consisting of an autograft, an allograft, demineralized bone matrix, and a collagen scaffold.

34. The composition of claim 21 for use in stimulating bone healing, accelerating bone healing, and/or improving bone healing in a subject.
